(19) United States
(12) Patent Application Publication
(10) Pub. No.: US 2008/0027012 A1
(43) Pub. Date: Jan. 31, 2008

(54) BRIDGED CARBAMATE MACROLIDES

(76) Inventors: Heejin Kim, Allston, MA (US); Ly Tam Phan, Quincy, MA (US); Yat Sun Or, Watertown, MA (US)

Correspondence Address: ELMORE PATENT LAW GROUP, PC, 209 MAIN STREET, N. CHELMSFORD, MA, 01863, US

(21) Appl. No.: 11/781,985

(22) Filed: Jul. 24, 2007

Related U.S. Application Data
(60) Provisional application No. 60/832,809, filed on Jul. 24, 2006.

(51) Int. Cl. A61K 31/7048; A61P 31/00; C07H 17/08

(52) U.S. Cl. 514/28; 536/7.1

(57) ABSTRACT

The present invention discloses compounds of formulae (I) and (II) or pharmaceutically acceptable salts, esters, or prodrugs thereof: 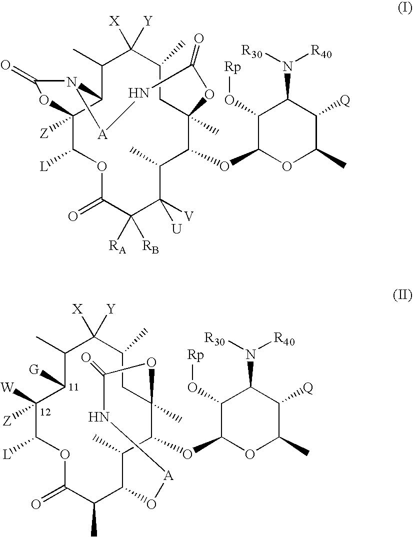 which exhibit antibacterial properties. The present invention further relates to pharmaceutical compositions comprising the aforementioned compounds for administration to a subject in need of antibiotic treatment. The invention also relates to methods of treating a bacterial infection in a subject by administering a pharmaceutical composition comprising the compounds of the present invention. The invention further includes process by which to make the compounds of the present invention.

### RELATED APPLICATION

This application claims the benefit of U.S. Provisional Patent Application No. 60/832,809, filed on Jul. 24, 2006. The entire teachings of the above application are incorporated herein by reference.

### FIELD OF THE INVENTION

The present invention relates to novel semisynthetic macrolides having antibacterial activity that are useful in the treatment and prevention of bacterial infections. More particularly, the invention relates to 3,6 and 6,11-bridged carbamate compounds, compositions containing such compounds and methods for using the same, as well as processes for making such compounds.

### BACKGROUND OF THE INVENTION

The spectrum of activity of macrolides, including erythromycin, covers most relevant bacterial species responsible for upper and lower respiratory tract infections. 14-membered ring macrolides are well known for their overall efficacy, safety and lack of serious side effects. Erythromycin however is quickly degraded into inactive products in the acidic medium of the stomach resulting in low bioavailability and gastrointestinal side effects. Improvement of erythromycin pharmacokinetics has been achieved through the synthesis of more acid-stable derivatives, for example, roxithromycin, clarithromycin, and the 15-membered ring macrolide azithromycin. However, all these drugs, including 16-membered ring macrolides, present several drawbacks. They are inactive against MLS_{B}-resistant streptococci (MLS_{B}=Macrolides-Lincosamides-type B Streptogramines) and with the exception of azithromycin, weakly active against Haemophilus influenzae. Furthermore, the resistance of Streptococcus pneumoniae to erythromycin has increased significantly in recent years (5% to above 40%). There is a high percentage of cross-resistance to penicillin among these isolates, with a worldwide epidemic spread of 10-40% in some areas.

There is, therefore, a clear need for new macrolides that overcome the problem of pneumococcal resistance, have good pharmacokinetic properties and acid stability while continuing to be active against H. influenzae. These new macrolides will be ideal candidates for drug development in the first line therapy of upper respiratory tract infections (“URTI”) and lower respiratory tract infections (“LRTI”).

Kashimura et al. have disclosed 6-O-methylerythromycin derivatives having a tricyclic basic nuclear structure in European Application 559896, published Nov. 11, 1991. Also, Asaka et al. have disclosed 5-O-desoaminylerythronolide derivatives containing a tricyclic carbamate structure in PCT Application WO 93/21200, published Apr. 22, 1992.

Recently erythromycin derivatives containing a variety of substituents at the 6-O position have been disclosed in U.S. Pat. Nos. 5,866,549 and 6,075,011 as well as PCT Application WO 00/78773. Furthermore, Ma et al. have described erythromycin derivatives with aryl groups tethered to the C-6 position in. J. Med. Chem., 44, pp 4137-4156 (2001). PCT application WO 97/10251, published Mar. 20, 1997, discloses intermediates useful for preparation of 6-O-methyl 3-descladinose erythromycin derivatives. U.S. Pat. Nos. 5,866,549 and 6,075,011, and PCT application WO 00/78773, published Dec. 28, 2000, disclose certain 6-O-substituted erythromycin derivatives.

PCT Application WO 03/095466 A1, published Nov. 20, 2003 and PCT Application WO 03/097659 A1, published Nov. 27, 2003 disclose a series of bicyclic erythromycin derivatives.

### SUMMARY OF THE INVENTION

The present invention provides a novel class of C6-C11 or C3-C6 bridged carbamate erythromycin compounds that possess antibacterial activity.

The compounds of the present invention are represented by formulae (I) and (II) as illustrated below: 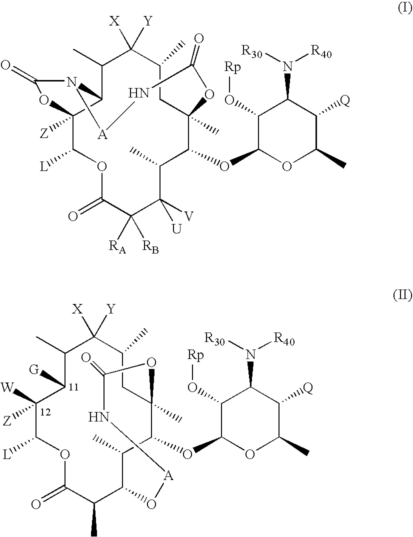
or the pharmaceutically acceptable salts, esters and prodrugs thereof,
wherein A is:
(a) —R₁—, where R₁ is substituted or unsubstituted —C₁-C₈ alkylene-, —C₂-C₈ alkenylene- or —C₂-C₈ alkynylene-, containing 0, 1, 2, or 3 heteroatoms selected from O, S or N;
(b) —R₁—(C═O)—R₂—, where R₂ is independently selected from R₁;
(c) —R₁—(C═N-M-R₃)—R₂—, where M is absent, O, NH, NH(CO), NH(CO)NH or NHSO₂; and R₃ is independently selected from the group consisting of:
   (1) hydrogen;
   (2) aryl; substituted aryl; heteroaryl; substituted heteroaryl; and
   (3) R₄, where R₄ is substituted or unsubstituted —C₁-C₆ alkyl, —C₂-C₆ alkenyl, or —C₂-C₆ alkynyl containing 0, 1, 2, or 3 heteroatoms selected from O, S or N; and
   (4) substituted or unsubstituted, saturated or unsaturated C₃-C₁₂ cycloalkyl containing 0, 1, 2, or 3 heteroatoms selected from O, S or N;
(d) —R₁—[C(OR₅)(OR₆)]—R₂—, where R₅ and R₆ are selected from the group consisting of C₁-C₁₂ alkyl, aryl or substituted aryl; or taken together —(CRxRy)ₘ-, where m is 2 or 3, Rx and Ry are independently R₃, alternatively, Rx and Ry can be taken together to form a fused heterocyclic or heteroaromatic ring;
(e) —R₁—[C(SR₅)(SR₆)]—R₂—; or
(f) —R₁—(C═CH—R₃)—R₂—;
   X and Y are each independently selected from the group consisting of:
(a) hydrogen;
(b) halogen;
(c) protected hydroxyl;
(d) —OC(O)R₃;
(e) —OR₃; and
(f) —NR₇R₈; wherein R₇ and R₈ are each independently selected from R₃; or R₇R₈ taken with the nitrogen atom to which they are connected form a 3 to 7-membered ring which may optionally contain a hetero function selected from the group consisting of —O—, —NR₃—, —S—, —S(O)—, and —S(O)₂—;
   Alternatively, X and Y taken together with the carbon atom to which they are attached is:
(a) C═O;
(b) C═N—OR₉, wherein R₉ is selected from the group consisting of:
   (1) hydrogen;
   (2) —CH₂—O—(CH₂)₂OCH₃;
   (3) —CH₂—O—(CH₂O)ₙCH₃, wherein n is as previously defined;
   (4) —R₄;
   (5) substituted and unsubstituted, saturated or unsaturated C₃-C₁₂ cycloalkyl;
   (6) substituted and unsubstituted heterocyclic;
   (7) C(O)—(C₃-C₁₂ cycloalkyl);
   (8) C(O)—R₃, wherein R₃ is as previously defined; and
   (9) —Si(Rₐ)(R_{b})(R_{c}), wherein Rₐ, R_{b} and R_{c} are each independently selected from the group consisting of C₁-C₁₂ alkyl, aryl and substituted aryl; or
   (10) C═N—O—C(R₉)(R₁₀)—O—R₁, wherein R₉ and R₁₀ taken together with the carbon atom to which they are attached form a C₃ to C₁₂ cycloalkyl group or each independently is selected from the group consisting of: hydrogen and C₁-C₁₂ alkyl; and R₁₁ is selected from the group consisting of:
      (i) —R₄;
      (ii) substituted and unsubstituted, saturated or unsaturated —C₃-C₁₂ cycloalkyl; and
      (iii) —Si(Rₐ)(R_{b})(R_{c}), wherein Rₐ, R_{b} and R_{c} are as previously defined;
         One U or V is hydrogen and the other is independently:
a) hydrogen;
b) hydroxyl;
c) protected hydroxyl;
d) —R₄; wherein R₄ is as previously defined
e) —OR₃;
f) —C(O)R₃;
g) —OC(O)R₃;
h) —S(O)ₙR₃; or
i) 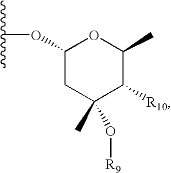
where R₉ is selected from the group consisting of hydrogen and methyl; and R₁₀ is:
   i. hydrogen;
   ii. hydroxyl or hydroxyl protecting group;
   iii. —R₄; or
   iv. —OR₄;
      Alternatively, U and V taken together with the carbon atom to which they are attached to form a carbonyl group;
      Each of R_{A} and R_{B} is independently:
a) hydrogen;
b) halogen; or
c) —R₄;
   W is selected from the group consisting of:
a) hydrogen;
b) —R₄; and
c) —OR₄;
   G is —OR₃; where R₃ is as previously defined;
   Alternatively, 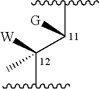
   moiety of formula (II) taken together is:
a) 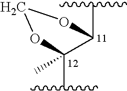
b) 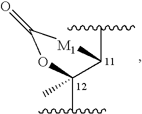
where M₁ is O or N-J-R₂₀, and where J is absent, O, NH, NH(CO), or N═CH; and R₂₀ is independent selected from R₃;
   R₃₀ and R₄₀ is independently selected from the group consisting of hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, saturated or unsaturated heterocyclic group; or can be taken together with the nitrogen atom to which they are attached to form a substituted or unsubstituted heterocyclic or heteroaromatic ring;
   Z is selected from the group consisting of hydrogen, azido, cyano, nitro, aldehyde, carboxylic acid, amide, a substituted or unsubstituted, saturated or unsaturated aliphatic group;
   Q is selected from the group consisting of:
(a) hydrogen;
(b) protected hydroxy; and
(c) OR₂₁, where R₂₁ is selected from the group consisting of:
   (i) —R₄; and
   (ii) substituted or unsubstituted, saturated or unsaturated —C₃-C₁₂ cycloalkyl containing 0, 1, 2, or 3 heteroatoms selected from O, S or N;
      L is —R₄;
      Rp is hydrogen, hydroxy protecting group or hydroxy prodrug group.

In another embodiment of the present invention there are disclosed pharmaceutical compositions comprising a therapeutically effective amount of a compound of the invention in combination with a pharmaceutically acceptable carrier or excipient. In yet another embodiment of the invention are methods of treating antibacterial infections in a subject in need of such treatment with said pharmaceutical compositions. Suitable carriers and formulations of the compounds of the present invention are disclosed.

### DETAILED DESCRIPTION OF THE INVENTION

In a first embodiment of the compounds of the present invention are compounds represented by formulae I and II as illustrated above, or a pharmaceutically acceptable salt, ester or prodrug thereof.

In a second embodiment of the compounds of the present invention are compounds represented by formula III as illustrated below, or a pharmaceutically acceptable salt, ester or prodrug thereof: 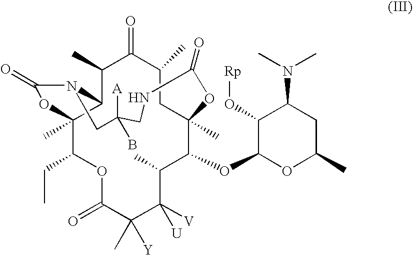
wherein A and B are independently selected from the group consisting of:
a) hydrogen;
b) hydroxyl;
c) activated hydroxyl;
d) N₃;
e) NH₂;
f) CN;
g) protected hydroxyl;
h) protected amino;
i) -T-R₃, where T is absent, O, S, S(O), SO₂, NH, NCH₃, NH(CO), NH(CO)NH or NHSO₂; and R₃ is as previously defined; and
j) 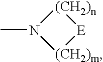
wherein E is absent, O, S, S(O), S(O)₂, NR₃, N(CO)R₃, NSO₂R₃, or CHR₃; n=1, 2, or 3; and m=2 or 3;
alternatively, A and B taken together with the carbon atom to which they are attached is:
   a) C═O;
   b) C(OR₅)(OR₆)], where R₅ and R₆ are selected from the group consisting of C₁-C₁₂ alkyl, aryl or substituted aryl; or taken together —(CRxRy)ₘ-, where m is 2 or 3, Rx and Ry are independently R₃, alternatively, Rx and Ry can be taken together to form a fused heterocyclic or heteroaromatic ring;
   c) C(SR₅)(SR₆);
   d) C═CHR₃;
   e) C═NRₐₚ; where Rₐₚ is amino protecting group
   f) C═N-M-R₃, where M is absent, O, NH, NH(CO), NH(CO)NH or NHSO₂;

and U, V, Y, and Rₚ are as previously defined.

In a third embodiment of the compounds of the present invention are compounds represented by formula IV as illustrated below, or a pharmaceutically acceptable salt, ester or prodrug thereof:

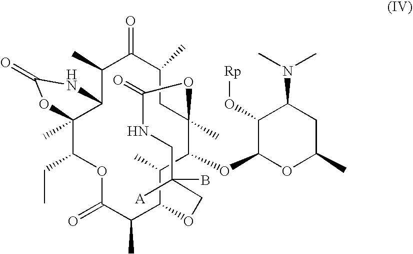

where A, B, and Rₚ are as previously defined.

In a fourth embodiment of the compounds of the present invention are compounds represented by formula V as illustrated below, or a pharmaceutically acceptable salt, ester or prodrug thereof:

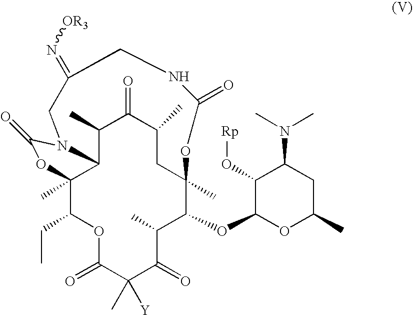

where Y, R₂ and Rₚ are as previously defined.

In a fifth embodiment of the compounds of the present invention are compounds represented by formula VI as illustrated below, or a pharmaceutically acceptable salt, ester or prodrug thereof:

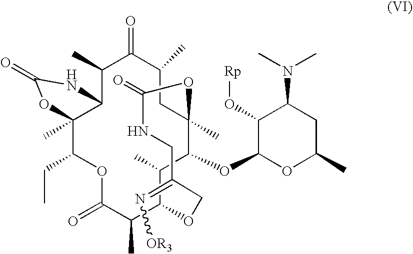

where R₃ and Rₚ are as previously defined.

Representative compounds according to the invention are those selected from the group consisting of:

(1) Compound of Formula III, wherein A and B taken together with the carbon atom they attached are C═CH₂, Y═U=hydrogen, V is 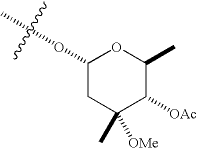
and Rp=Ac;
(2) Compound of Formula III, wherein A and B taken together with the carbon atom they attached are C═CH₂, Y═U=hydrogen, V═OH, and Rp=Ac;
(3) Compound of Formula III, wherein A and B taken together with the carbon atom they attached are C═CH₂, U and V taken together with the carbon atom they attached are C═O, and Rp=Ac;
(4) Compound of Formula III, wherein A and B taken together with the carbon atom they attached are C═O, U and V taken together with the carbon atom they attached are C═O, and Rp=Ac;
(5) Compound of Formula IV, wherein A and B taken together with the carbon atom they attached are C═O, U and V taken together with the carbon atom they attached are C═O, and Rp=Ac.

Further representative compounds according to the invention are those selected from the group consisting of:
Compounds (6)-(17) of the Formula A:

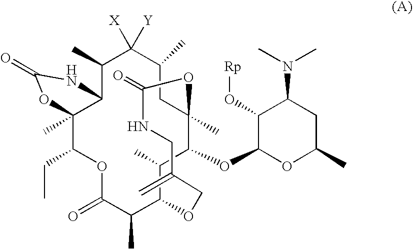

wherein X and Y taken together with the carbon to which they are attached (CXY) and Rp are delineated for each example in TABLE 1:

TABLE 1

| Compound | CXY | Rp |
|---|---|---|
| (6) | C=NOAc | Ac |
| (7) | C=NOBz | Bz |
| (8) | C═NOSiEt₃ | SiEt₃ |
| (9) | 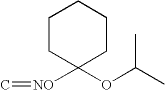 | Ac |
| (10) | 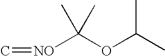 | Ac |
| (11) | C═NOH | H |
| (12) | C═NH | H |
| (13) | CH(NH₂) | H |
| (14) | C═O | H |
| (15) | C═O | Ac |
| (16) | C═NAc | Ac |
| (17) | C═NAc | H |

Further representative species of the present invention are:
Compounds (18)-(165) of the Formula (B):

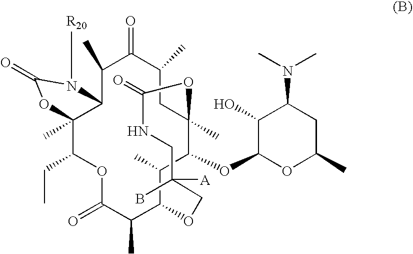

wherein A and B taken together with the carbon to which they are attached (CAB), and R₂₀ are delineated for each example in Table 2.

TABLE 2

| Compound | R₂₀ | CAB |
|---|---|---|
| (18) | H | C=O |
| (19) | H | C=O |
| (20) | 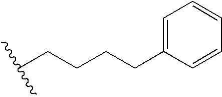 | C=CH₂ |
| (21) | 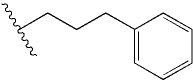 | C═CH₂ |
| (22) | 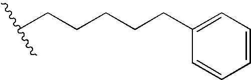 | C═CH₂ |
| (23) | 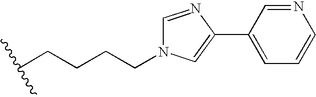 | C═CH₂ |
| (24) | 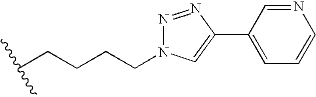 | C═CH₂ |
| (25) | 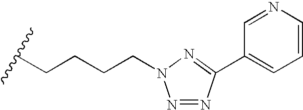 | C═CH₂ |
| (26) | 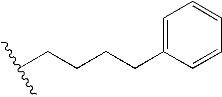 | C═O |
| (27) | 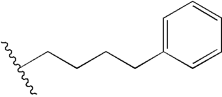 | CHCH₃ |
| (28) | 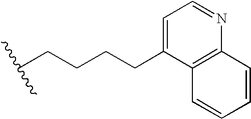 | C═CH₂ |
| (29) | H | C═NO[CH₂Ph] |
| (30) | H | C═NO[(CH₂)₂Ph] |
| (31) | H | C═NO[(CH₂)₃Ph] |
| (32) | H | C═NO[(CH₂)₄Ph] |
| (33) | H | C═NO[Ph] |
| (34) | H | C═NO[(CH₂)₅Ph] |
| (35) | H | 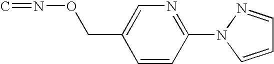 |
| (36) | H | 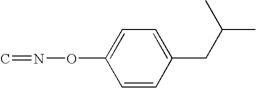 |
| (37) | H | 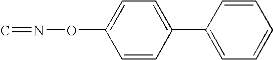 |
| (38) | H | 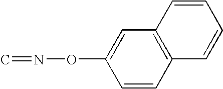 |
| (39) | H | 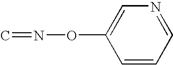 |
| (40) | H | C═NNH[Ph] |
| (41) | H | CH(CH₂)₃Ph |
| (42) | H | CHCH₃ |
| (43) | H | CHOH |
| (44) | H | 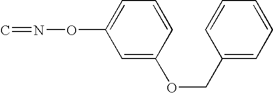 |
| (45) | H | 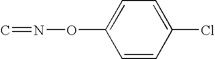 |
| (46) | H | 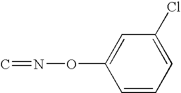 |
| (47) | H | 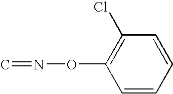 |
| (48) | H | 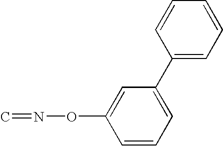 |
| (49) | H | 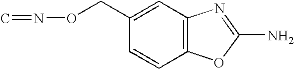 |
| (50) | H | 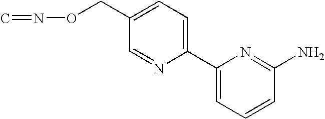 |
| (51) | 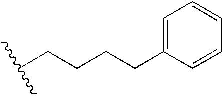 | C═NO [Ph] |
| (52) | 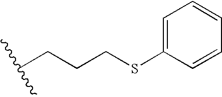 | C═CH₂ |
| (53) | 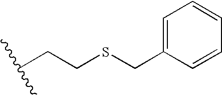 | C═CH₂ |
| (54) | H | 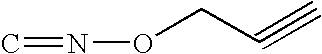 |
| (55) | H | 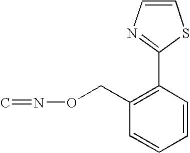 |
| (56) | H | 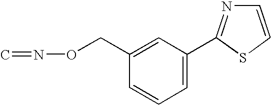 |
| (57) | H | 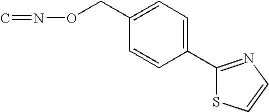 |
| (58) | H | 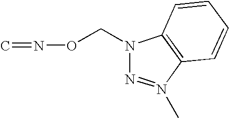 |
| (59) | H | 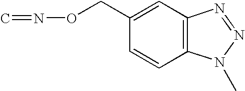 |
| (60) | H | 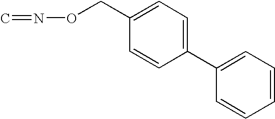 |
| (61) | H | 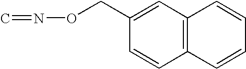 |
| (62) | H | 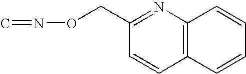 |
| (63) | H | 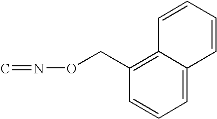 |
| (64) | H | 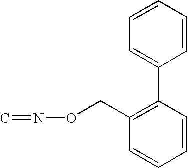 |
| (65) | H | 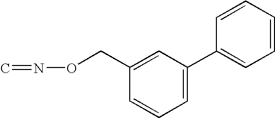 |
| (66) | H | 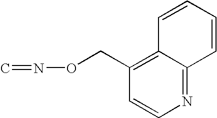 |
| (67) | H | 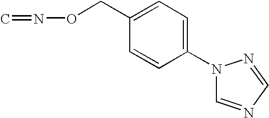 |
| (68) | H | 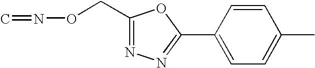 |
| (69) | H | 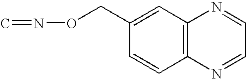 |
| (70) | H | 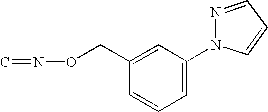 |
| (71) | H | 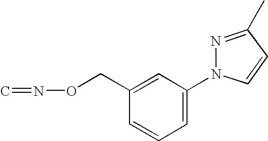 |
| (72) | H | 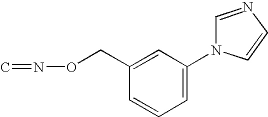 |
| (73) | H | 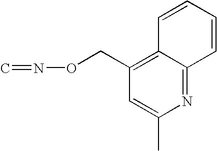 |
| (74) | H | 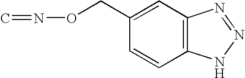 |
| (75) | H | 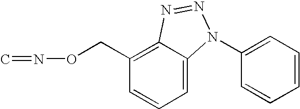 |
| (76) | H | 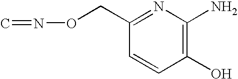 |
| (77) | H | 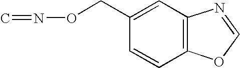 |
| (78) | H | 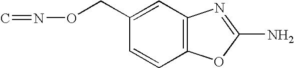 |
| (79) | H | 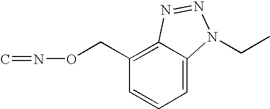 |
| (80) | H | C═N—OH |
| (81) | H | 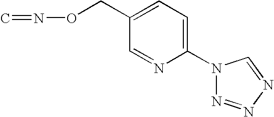 |
| (82) | H | 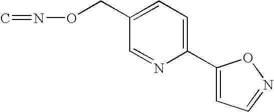 |
| (83) | H | 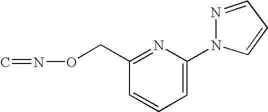 |
| (84) | H | 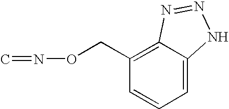 |
| (85) | H | 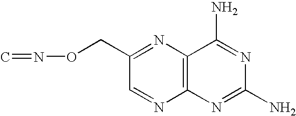 |
| (86) | H | 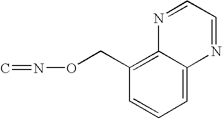 |
| (87) | H | 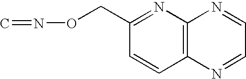 |
| (88) | H | 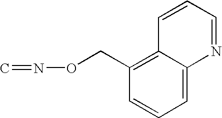 |
| (89) | H | 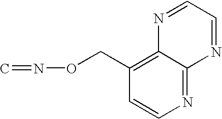 |
| (90) | H | 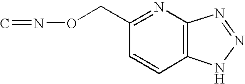 |
| (91) | H | 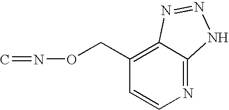 |
| (92) | H | 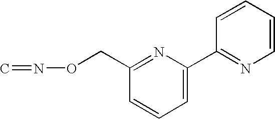 |
| (93) | H | 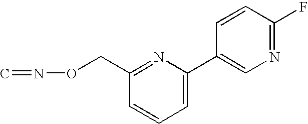 |
| (94) | H | 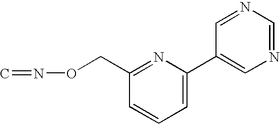 |
| (95) | H | 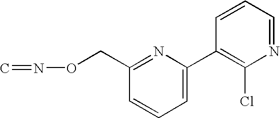 |
| (96) | H | 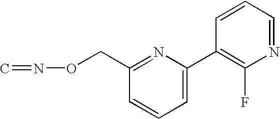 |
| (97) | H | 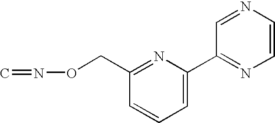 |
| (98) | H | 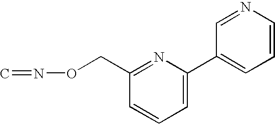 |
| (99) | H | 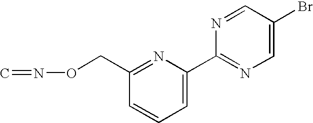 |
| (100) | H | 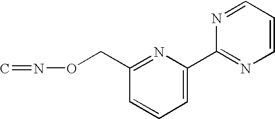 |
| (101) | H | 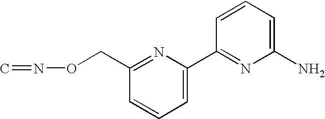 |
| (102) | H | 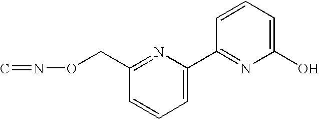 |
| (103) | H | 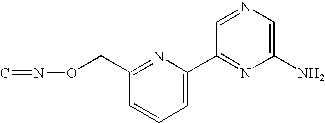 |
| (104) | H | 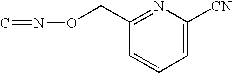 |
| (105) | H | 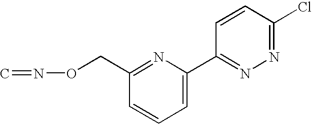 |
| (106) | H | 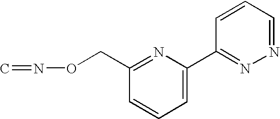 |
| (107) | H | 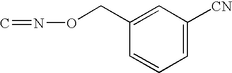 |
| (108) | H | 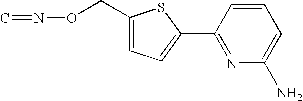 |
| (109) | H | 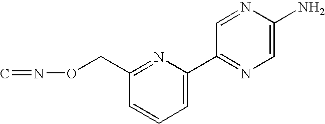 |
| (110) | H | 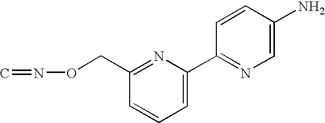 |
| (111) | H | 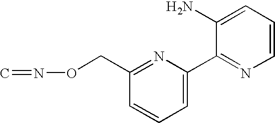 |
| (112) | H | 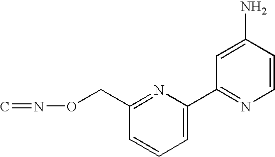 |
| (113) | H | 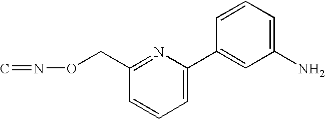 |
| (114) | H | 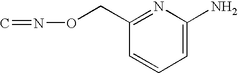 |
| (115) | H | 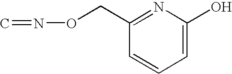 |
| (116) | H | 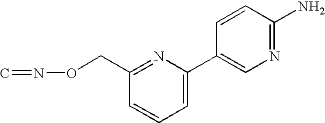 |
| (117) | H | 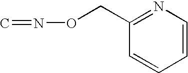 |
| (118) | H | 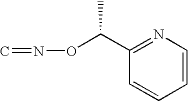 |
| (119) | H | 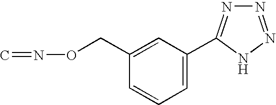 |
| (120) | CH₃ | C═CH₂ |
| (121) | CH₃ | C═O |
| (122) | CH₃ | 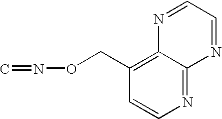 |
| (123) | CH₃ | 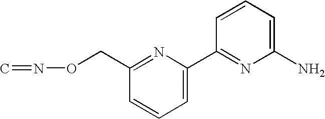 |
| (124) | H | 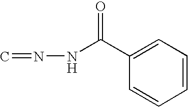 |
| (125) | H | 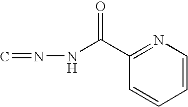 |
| (126) | H | 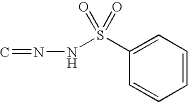 |
| (127) | H | 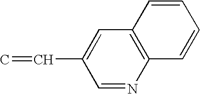 |
| (128) | H | 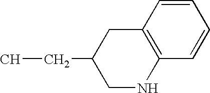 |
| (129) | H | 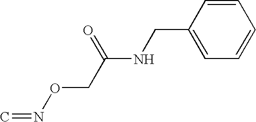 |
| (130) | H | 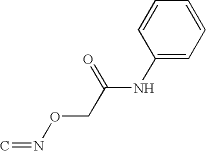 |
| (131) | H | 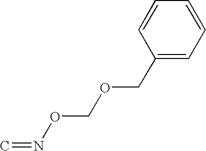 |
| (132) | H | 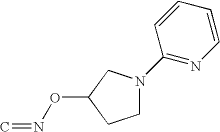 |
| (133) | H | 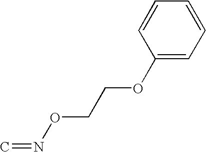 |
| (134) | H | 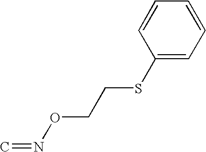 |
| (135) | H | 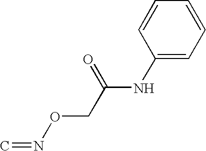 |
| (136) | H | 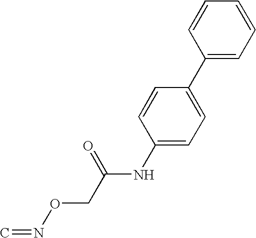 |
| (137) | H | 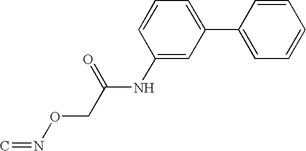 |
| (138) | H | 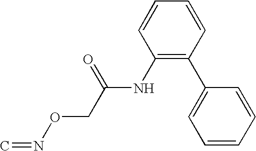 |
| (139) | H | 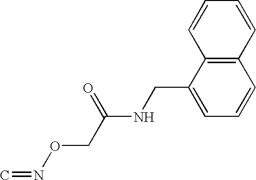 |
| (140) | H | 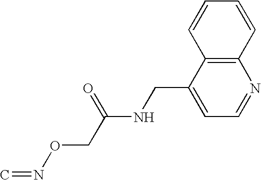 |
| (141) | H | 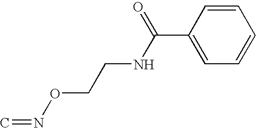 |
| (142) | H | 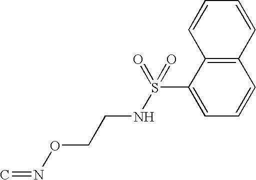 |
| (143) | H | 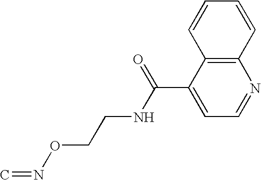 |
| (144) | H | 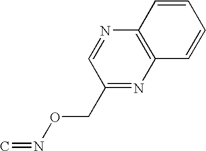 |
| (145) | H | 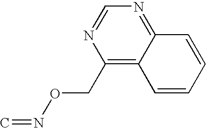 |
| (146) | H | 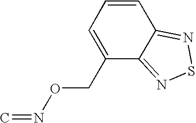 |
| (147) | H | 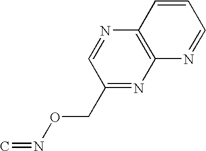 |
| (148) | H | 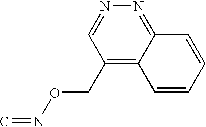 |
| (149) | H | 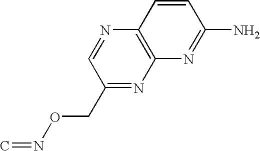 |
| (150) | H | 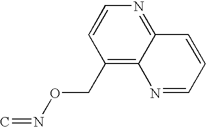 |
| (151) | H | 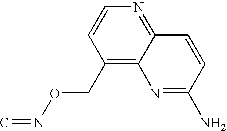 |
| (152) | H | 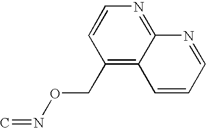 |
| (153) | H | 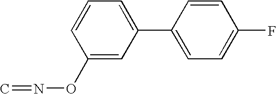 |
| (154) | H | 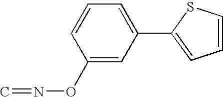 |
| (155) | H | 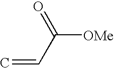 |
| (156) | H | 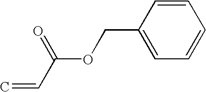 |
| (157) | H | 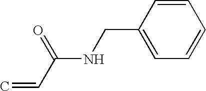 |
| (158) | H | 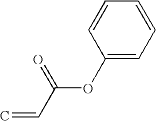 |
| (159) | H | 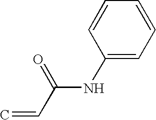 |
| (160) | H | 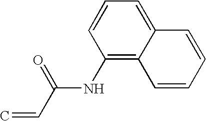 |
| (161) | H | 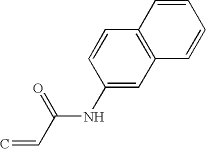 |
| (162) | H | 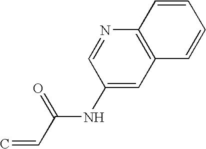 |
| (163) | H | 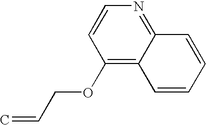 |
| (164) | H | 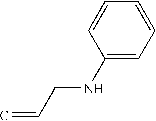 |
| (165) | H | 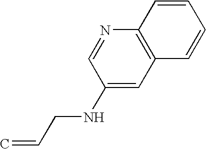 |

Further representative species of the present invention are:
Compounds (166)-(232) of the Formula (B):

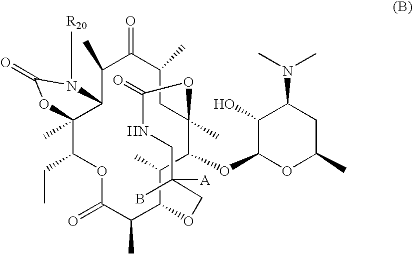

wherein A, B, and R₂₀ are delineated for each example in Table 3.

TABLE 3

| Compound | R₂₀ | A | B |
|---|---|---|---|
| (166) | H | 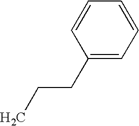 | OH |
| (167) | H | 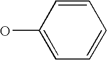 | H |
| (168) | H | 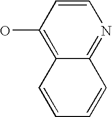 | H |
| (169) | H | 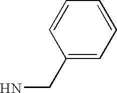 | H |
| (170) | H | 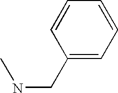 | H |
| (171) | H | 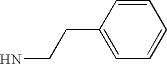 | H |
| (172) | H | 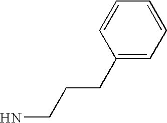 | H |
| (173) | H | 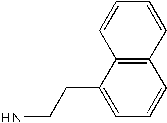 | H |
| (174) | H | 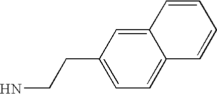 | H |
| (175) | H | 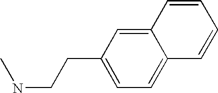 | H |
| (176) | H | 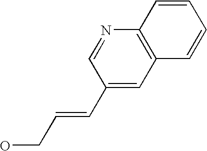 | H |
| (177) | H | 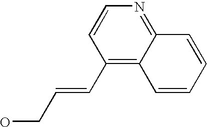 | H |
| (178) | H | OH (R—) | H |
| (179) | H | 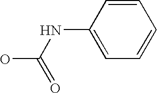 | H |
| (180) | H | 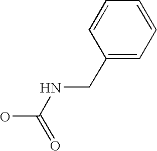 | H |
| (181) | H | OSO₂CH₃ (S—) | H |
| (182) | H | N₃ | H |
| (183) | H | NH₂ | H |
| (184) | H | 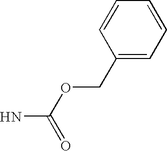 | H |
| (185) | H | 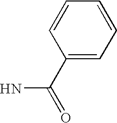 | H |
| (186) | H | 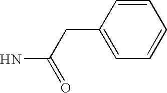 | H |
| (187) | H | 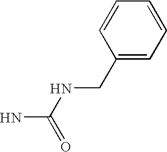 | H |
| (188) | H | 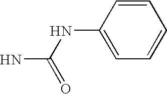 | H |
| (189) | H | 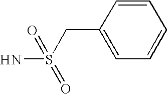 | H |
| (190) | H | 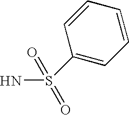 | H |
| (191) | H | 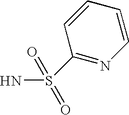 | H |
| (192) | H | 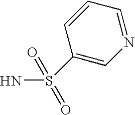 | H |
| (193) | H | 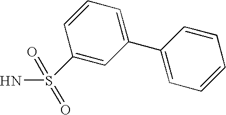 | H |
| (194) | H | 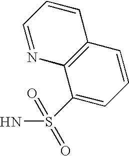 | H |
| (195) | H | 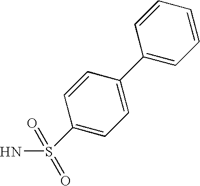 | H |
| (196) | H | 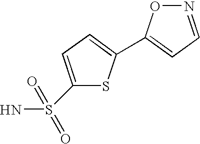 | H |
| (197) | H | 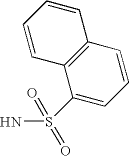 | H |
| (198) | H | 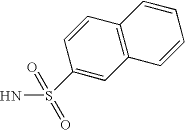 | H |
| (199) | H | 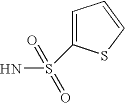 | H |
| (200) | H | 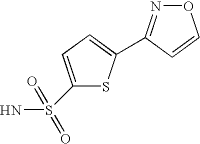 | H |
| (201) | H | 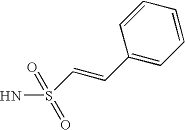 | H |
| (202) | H | 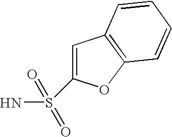 | H |
| (203) | H | 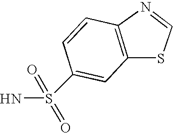 | H |
| (204) | H | 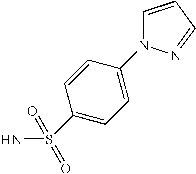 | H |
| (205) | H | 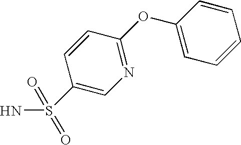 | H |
| (206) | H | 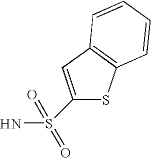 | H |
| (207) | H | 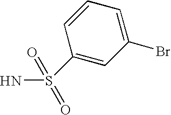 | H |
| (208) | H | 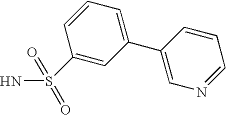 | H |
| (209) | H | 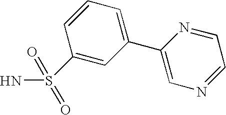 | H |
| (210) | H | 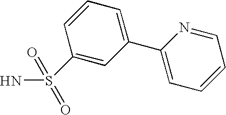 | H |
| (211) | H | 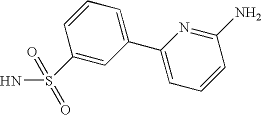 | H |
| (212) | H | 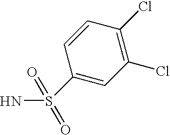 | H |
| (213) | H | 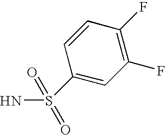 | H |
| (214) | H | 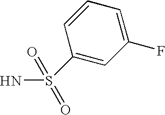 | H |
| (215) | H | 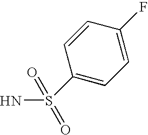 | H |
| (216) | H | 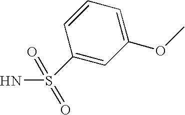 | H |
| (217) | H | 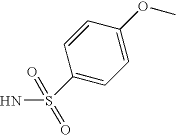 | H |
| (218) | H | 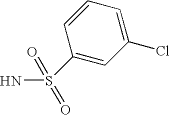 | H |
| (219) | H | 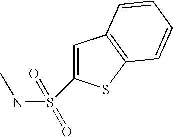 | H |
| (220) | H | 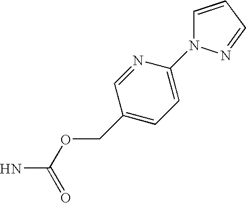 | H |
| (221) | H | 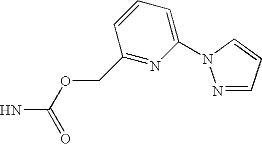 | H |
| (222) | H | 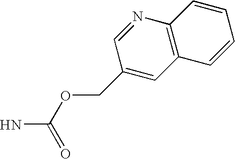 | H |
| (223) | H | 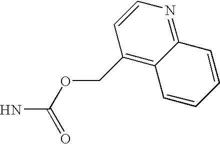 | H |
| (224) | H | 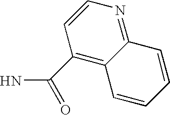 | H |
| (225) | H | 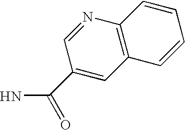 | H |
| (226) | H | 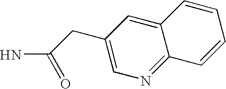 | H |
| (227) | H | 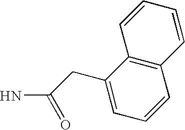 | H |
| (228) | H | 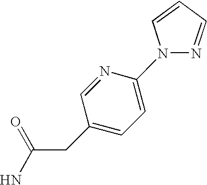 | H |
| (229) | H | 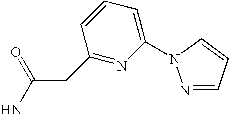 | H |
| (230) | H | 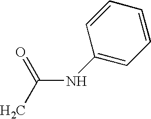 | H |
| (231) | H | 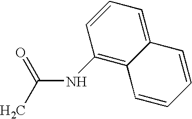 | H |
| (232) | H | 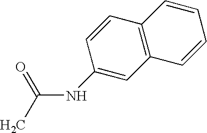 | H |

Further representative species of the present invention are:
Compounds (233)-(394) of the Formula C:

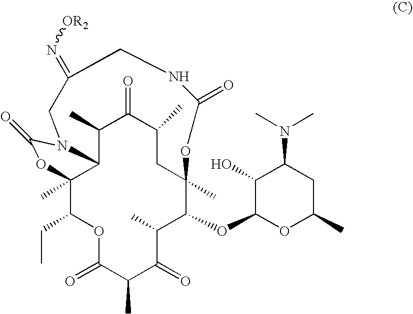

wherein R₂ is delineated for each example in Table 4.

TABLE 4

| Compound | R₂ |
|---|---|
| (233) | 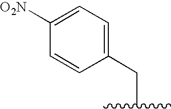 |
| (234) | 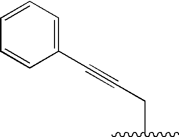 |
| (235) | 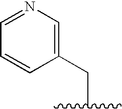 |
| (236) | 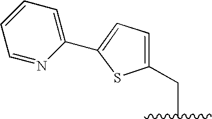 |
| (237) | 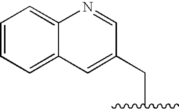 |
| (238) | 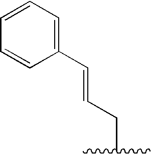 |
| (239) | 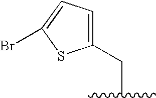 |
| (240) | 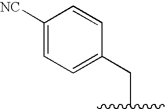 |
| (241) | 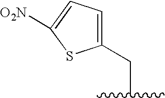 |
| (242) | 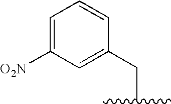 |
| (243) | 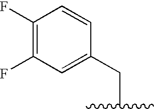 |
| (244) | 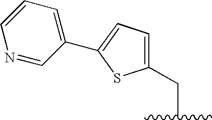 |
| (245) | 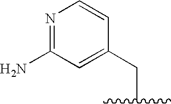 |
| (246) | 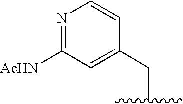 |
| (247) | 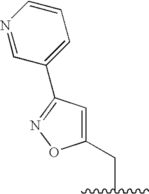 |
| (248) | 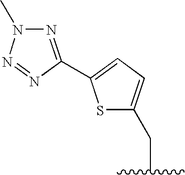 |
| (249) | 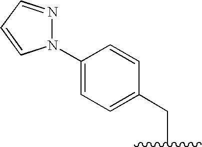 |
| (250) | 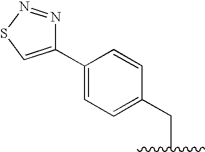 |
| (251) | 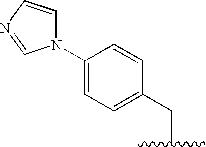 |
| (252) | 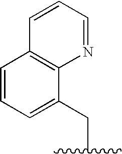 |
| (253) | 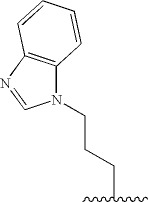 |
| (254) | 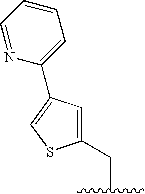 |
| (255) | 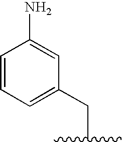 |
| (256) | 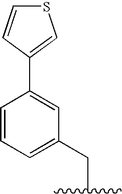 |
| (257) | 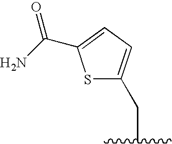 |
| (258) | 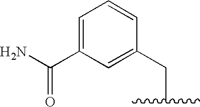 |
| (259) | 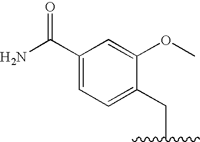 |
| (260) | 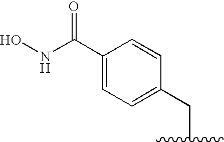 |
| (261) | 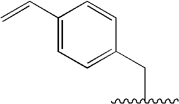 |
| (262) | 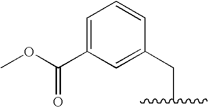 |
| (263) | 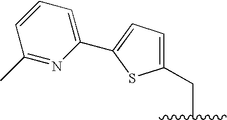 |
| (264) | 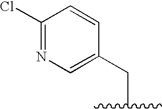 |
| (265) | 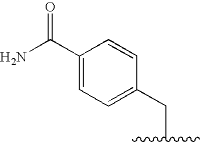 |
| (266) | 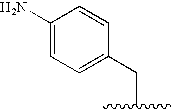 |
| (267) | 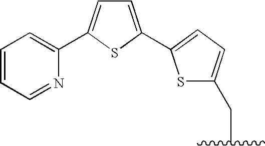 |
| (268) | 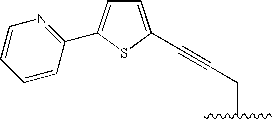 |
| (269) | 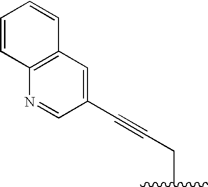 |
| (270) | 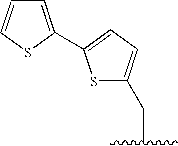 |
| (271) | 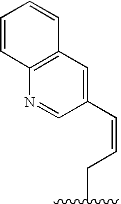 |
| (272) | 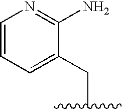 |
| (273) | 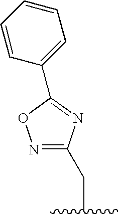 |
| (274) | 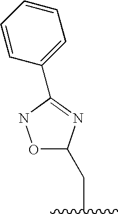 |
| (275) | 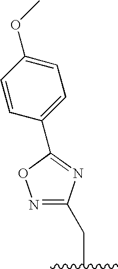 |
| (276) | 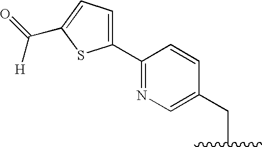 |
| (277) | 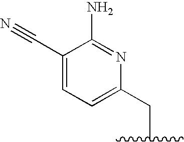 |
| (278) | 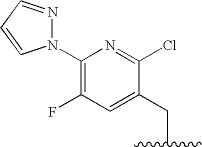 |
| (279) | 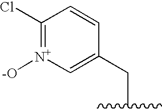 |
| (280) | 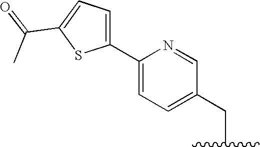 |
| (281) | 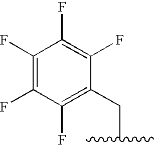 |
| (282) | 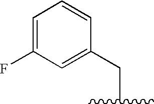 |
| (283) | 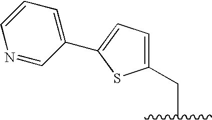 |
| (284) | 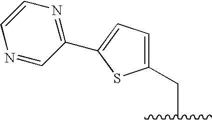 |
| (285) | 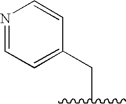 |
| (286) | 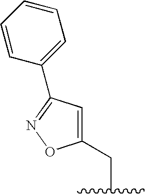 |
| (287) | 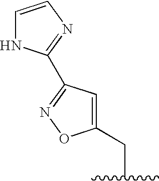 |
| (288) | 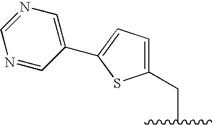 |
| (289) | 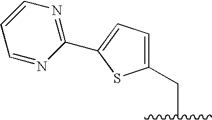 |
| (290) | 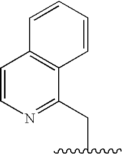 |
| (291) | 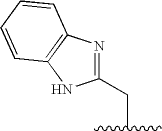 |
| (292) | 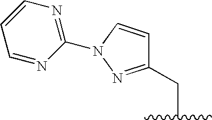 |
| (293) | 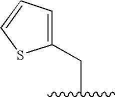 |
| (294) | 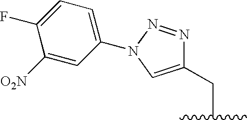 |
| (295) | 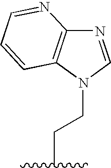 |
| (296) | 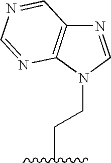 |
| (297) | 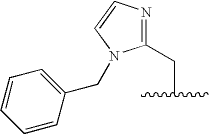 |
| (298) | 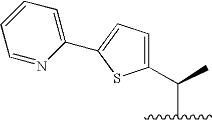 |
| (299) | 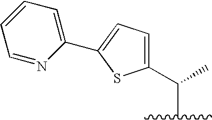 |
| (300) | 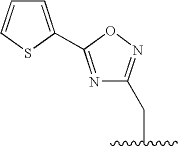 |
| (301) | 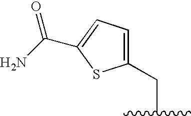 |
| (302) | 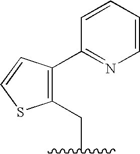 |
| (303) | 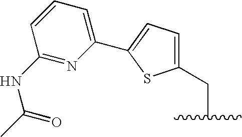 |
| (304) | 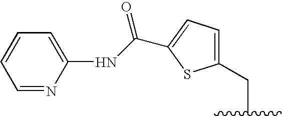 |
| (305) | 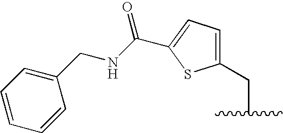 |
| (306) | 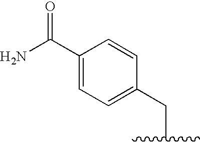 |
| (307) | 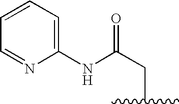 |
| (308) | 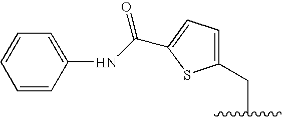 |
| (309) | 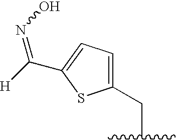 |
| (310) | 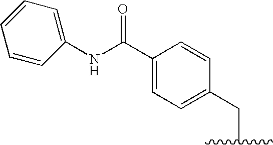 |
| (311) | 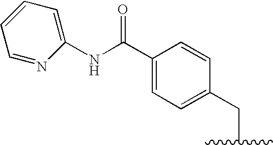 |
| (312) | 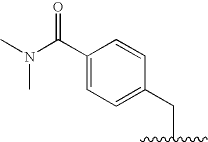 |
| (313) | 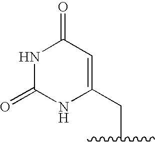 |
| (314) | 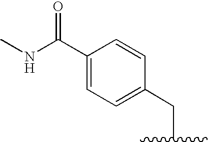 |
| (315) | 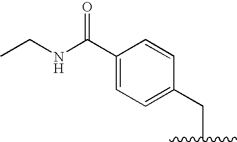 |
| (316) | 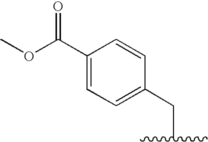 |
| (317) | 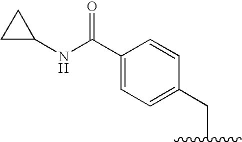 |
| (318) | 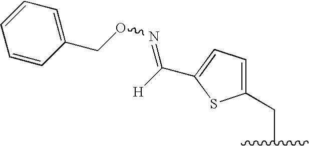 |
| (319) | 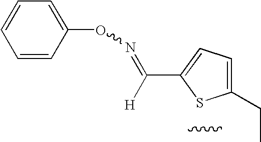 |
| (320) | 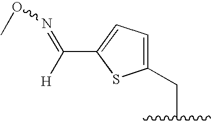 |
| (321) | 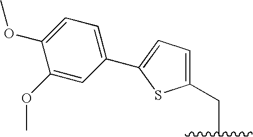 |
| (322) | 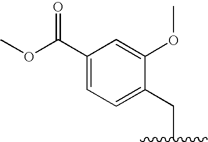 |
| (323) | 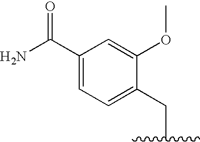 |
| (324) | 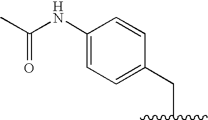 |
| (325) | 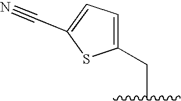 |
| (326) | 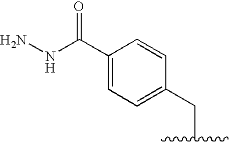 |
| (327) | 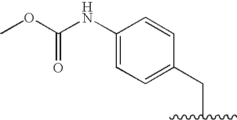 |
| (328) | 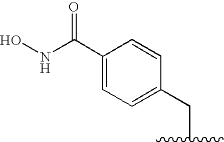 |
| (329) | 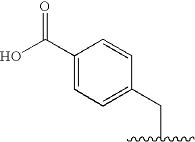 |
| (330) | 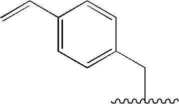 |
| (331) | 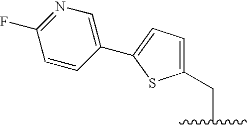 |
| (332) | 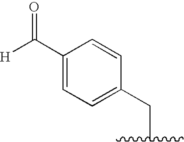 |
| (333) | 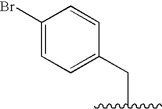 |
| (334) | 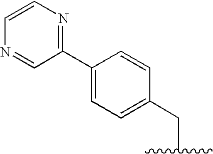 |
| (335) | 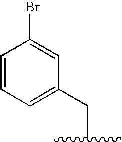 |
| (336) | 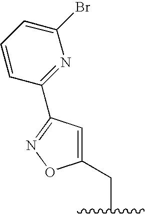 |
| (337) | 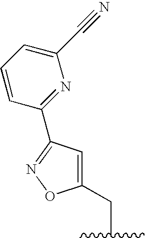 |
| (338) | 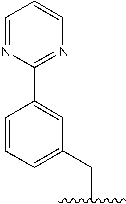 |
| (339) | 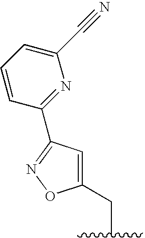 |
| (340) | 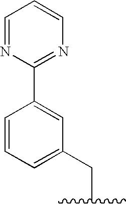 |
| (341) | 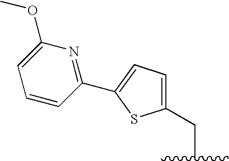 |
| (342) | 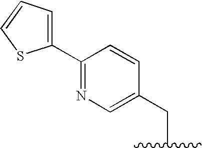 |
| (343) | 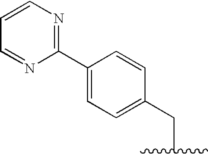 |
| (344) | 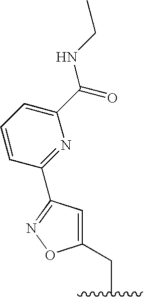 |
| (345) | 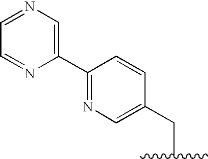 |
| (346) | 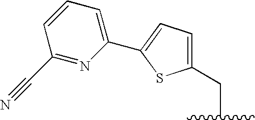 |
| (347) | 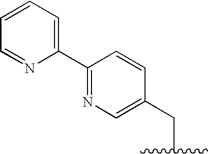 |
| (348) | 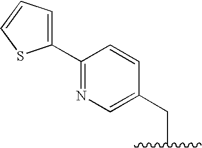 |
| (349) | 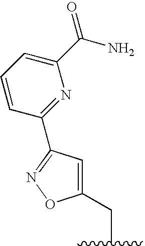 |
| (350) | 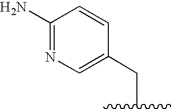 |
| (351) | 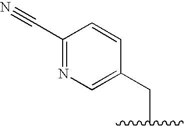 |
| (352) | 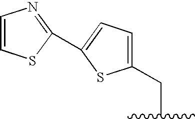 |
| (353) | 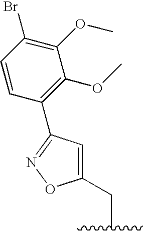 |
| (354) | 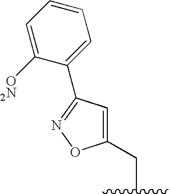 |
| (355) | 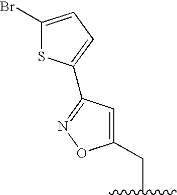 |
| (356) | 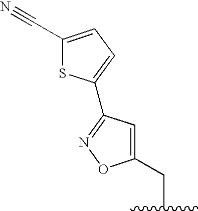 |
| (357) | 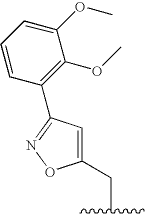 |
| (358) | 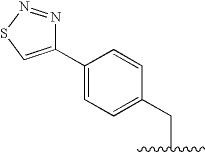 |
| (359) | 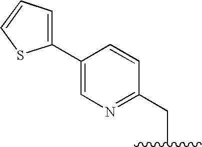 |
| (360) | 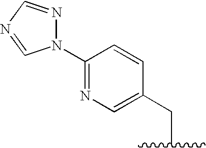 |
| (361) | 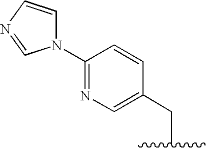 |
| (362) | 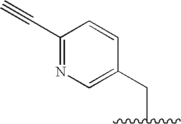 |
| (363) | 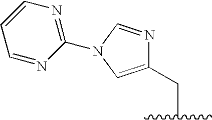 |
| (364) | 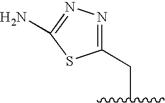 |
| (365) | 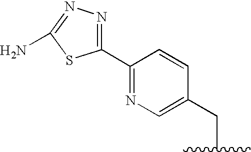 |
| (366) | 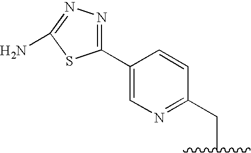 |
| (367) | 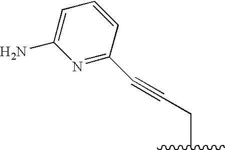 |
| (368) | 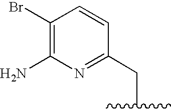 |
| (369) | 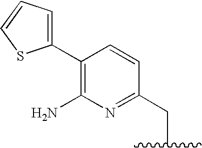 |
| (370) | 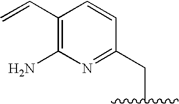 |
| (371) | 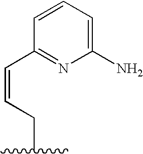 |
| (372) | 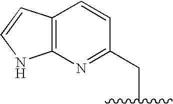 |
| (373) | 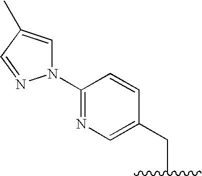 |
| (374) | 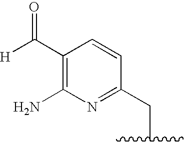 |
| (375) | 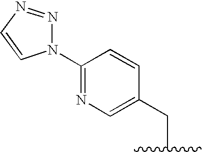 |
| (376) | 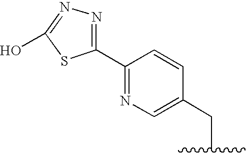 |
| (377) | 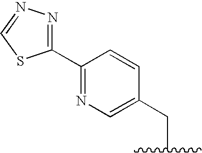 |
| (378) | 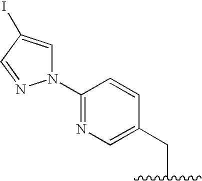 |
| (379) | 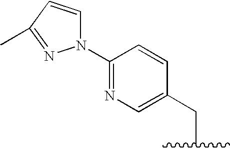 |
| (380) | 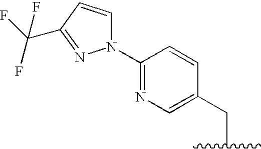 |
| (381) | 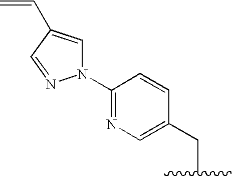 |
| (382) | 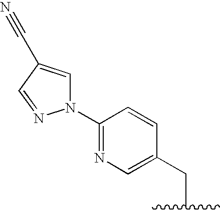 |
| (383) | 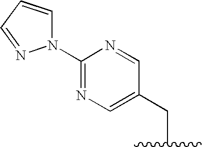 |
| (384) | 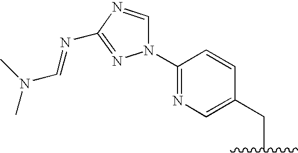 |
| (385) | 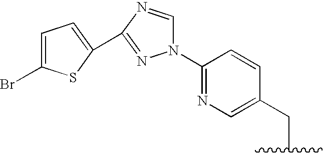 |
| (386) | 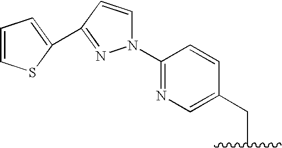 |
| (387) | 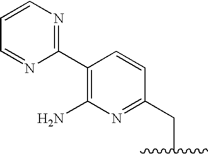 |
| (388) | 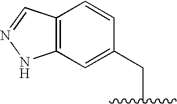 |
| (389) | 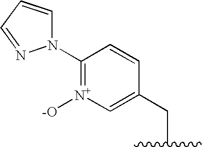 |
| (390) | 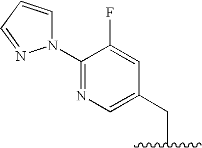 |
| (391) | 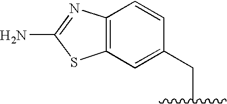 |
| (392) | 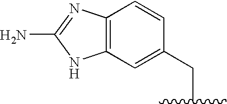 |
| (393) | 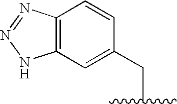 |
| (394) | 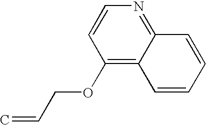 |

A further embodiment of the present invention includes pharmaceutical compositions comprising any single compound delineated herein, or a pharmaceutically acceptable salt, ester, or prodrug thereof, with a pharmaceutically acceptable carrier or excipient.

Yet another embodiment of the present invention is a pharmaceutical composition comprising a combination of two or more compounds delineated herein, or a pharmaceutically acceptable salt, ester, or prodrug thereof, with a pharmaceutically acceptable carrier or excipient.

Yet a further embodiment of the present invention is a pharmaceutical composition comprising any single compound delineated herein in combination with one or more antibiotics known in the art, or a pharmaceutically acceptable salt, ester, or prodrug thereof, with a pharmaceutically acceptable carrier or excipient.

In addition, the present invention contemplates processes of making any compound delineated herein via any synthetic method delineated herein.

### DEFINITIONS

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system having one or two aromatic rings including, but not limited to, phenyl, naphthyl, tetrahydronaphthyl, indanyl, idenyl and the like.

The term “heteroaryl,” as used herein, refers to a mono- or polycyclic (e.g. bi-, or tri-cyclic or more) aromatic radical or ring having from five to ten ring atoms of which one or more ring atom is selected from, for example, S, O and N; zero, one or two ring atoms are additional heteroatoms independently selected from, for example, S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

In accordance with the invention, any of the aryls, substituted aryls, heteroaryls and substituted heteroaryls described herein, can be any aromatic group. Aromatic groups can be substituted or unsubstituted.

The terms “C₁-C₆ alkyl,” or “C₁-C₁₂ alkyl,” as used herein, refer to saturated, straight- or branched-chain hydrocarbon radicals containing between one and six, or one and twelve carbon atoms, respectively. Examples of C₁-C₆ alkyl radicals include, but are not limited to, methyl, ethyl, propyl, isopropyl, n-butyl, tert-butyl, neopentyl and n-hexyl radicals; and examples of C₁-C₁₂ alkyl radicals include, but are not limited to, ethyl, propyl, isopropyl, n-hexyl, octyl, decyl, dodecyl radicals.

The term “C₂-C₆ alkenyl,” as used herein, denotes a monovalent group derived from a hydrocarbon moiety containing from two to six carbon atoms having at least one carbon-carbon double bond by the removal of a single hydrogen atom. Alkenyl groups include, but are not limited to, for example, ethenyl, propenyl, butenyl, 1-methyl-2-buten-1-yl, and the like.

The term “C₂-C₆ alkynyl,” as used herein, denotes a monovalent group derived from a hydrocarbon moiety containing from two to six carbon atoms having at least one carbon-carbon triple bond by the removal of a single hydrogen atom. Representative alkenyl groups include, but are not limited to, for example, ethynyl, 1-propynyl, 1-butynyl, and the like.

The term “C₁-C₈ alkylene,” as used herein, refer to saturated, straight- or branched-chain hydrocarbon containing between one and eight. Alkylene groups include, but are not limited to, ethylene, propylene, butylene, 3-methyl-pentylene, and 5-ethyl-hexylene.

The term “C₂-C₈ alkenylene,” as used herein, denotes a divalent group derived from a straight chain or branch hydrocarbon moiety containing from two to eight carbon atoms having at least one carbon-carbon double bond. Alkenylene groups include, but are not limited to, for example, ethenylene, 2-propenylene, 2-butenylene, 1-methyl-2-buten-1-ylene, and the like.

The term “C₂-C₈ alkynylene,” as used herein, denotes a divalent group derived from a straight chain or branch hydrocarbon moiety containing from two to eight carbon atoms having at least one carbon-carbon triple bond. Representative alkynylene groups include, but are not limited to, for example, propynylene, 1-butynylene, 2-methyl-3-hexynylene, and the like.

The term “C₃-C₁₂-cycloalkyl,” as used herein, denotes a monovalent group derived from a monocyclic or polycyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl.

It is understood that any alkyl, alkenyl, alkynyl and cycloalkyl moiety described herein can also be an aliphatic group, an alicyclic group or a heterocyclic group. An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl. Such alicyclic groups may be further substituted.

The term “heterocyclic” as used herein, refers to a non-aromatic 5-, 6- or 7-membered ring or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl. Such heterocyclic groups may be further substituted.

The terms “substituted aryl’, “substituted heteroaryl,” “substituted C₁-C₆ alkyl,” “substituted C₁-C₁₂ alkyl,” “substituted C₂-C₆ alkenyl,” “substituted C₂-C₆ alkynyl,” “substituted C₁-C₈ alkylene,” “substituted C₂-C₈ alkenylene,” “substituted C₂-C₈ alkynylene,” “substituted aliphatic,”,” or “substituted C₃-C₁₂ cycloalkyl,” as used herein, refer to aryl, heteroaryl, C₁-C₆ alkyl, C₁-C₁₂ alkyl, C₂-C₆ alkenyl, C₂-C₆ alkynyl, C₁-C₈ alkylene, C₂-C₈ alkenylene, aliphatic, or C₃-C₁₂ cycloalkyl groups as previously defined, substituted by independent replacement of one, two, or three or more of the hydrogen atoms thereon with substituents including, but not limited to, —F, —Cl, —Br, —I, —OH, protected hydroxyl, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₃-C₁₋₂-alkyl, —NHCO₂—C₂-C₁₂-alkenyl, —NHCO₂—C₂-C₁₂-alkenyl, —NHCO₂—C₃-C₁₂-cycloalkyl, —NHCO₂— aryl, —NHCO₂— heteroaryl, —NHCO₂-heterocycloalkyl, —NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH— aryl, —SO₂NH— heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl. It is understood that the aryls, heteroaryls, alkyls, and the like can be further substituted.

The term “halogen,” as used herein, refers to an atom selected from fluorine, chlorine, bromine and iodine.

The term “hydroxy activating group”, as used herein, refers to a labile chemical moiety which is known in the art to activate a hydroxyl group so that it will depart during synthetic procedures such as in a substitution or an elimination reactions. Examples of hydroxyl activating group include, but not limited to, mesylate, tosylate, triflate, p-nitrobenzoate, phosphonate and the like.

The term “activated hydroxy”, as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group”, as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al, Vol. II, in the Techniques of Chemistry Series, John Wiley & Sons, NY, 1986.

The term “protogenic organic solvent,” or “protic solvent’ as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This invention also encompasses pharmaceutical compositions containing, and methods of treating bacterial infections through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

As used herein, unless otherwise indicated, the term “bacterial infection(s)” or “protozoa infections”; includes, but is not limited to, bacterial infections and protozoa infections that occur in mammals, fish and birds as well as disorders related to bacterial infections and protozoa infections that may be treated or prevented by administering antibiotics such as the compounds of the present invention. Such bacterial infections and protozoa infections and disorders related to such infections include, but are not limited to, the following: pneumonia, otitis media, sinusitus, bronchitis, tonsillitis, cystic fibrosis and mastoiditis related to infection by Streptococcus pneumoniae, Haemophilus influenzae, Moraxella catarrhalis, Staphylococcus aureus, Peptostreptococcus spp, or Pseudomonas spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S. and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp. odontogenic infection related to infection by viridans streptococci; persistent cough related to infection by Bordetella pertussis; gas gangrene related to infection by Clostridium perfringens or Bacteroides spp.; Skin infection by S. aureus, Propionibacterium acne; atherosclerosis related to infection by Helicobacter pylori or Chlamydia pneumoniae; or the like.

Bacterial infections and protozoa infections and disorders related to such infections that may be treated or prevented in animals include, but are not limited to, the following: bovine respiratory disease related to infection by P. haemolytica, P. multocida, Mycoplasma bovis, or Bordetella spp.; cow enteric disease related to infection by E. coli or protozoa (i.e., coccidia, cryptosporidia, etc.), dairy cow mastitis related to infection by S. aureus, S. uberis, S. agalactiae, S. dysgalactiae, Klebsiella spp., Corynebacterium, or Enterococcus spp.; swine respiratory disease related to infection by A. pleuropneumoniae, P. multocida, or Mycoplasma spp.; swine enteric disease related to infection by E. coli, Lawsonia intracellularis, Salmonella spp., or Serpulina hyodyisinteriae; cow footrot related to infection by Fusobacterium spp.; cow metritis related to infection by E. coli; cow hairy warts related to infection by Fusobacterium necrophorum or Bacteroides nodosus; cow pink-eye related to infection by Moraxella bovis, cow premature abortion related to infection by protozoa (i.e. neosporium); urinary tract infection in dogs and cats related to infection by E. coli; skin and soft tissue infections in dogs and cats related to infection by S. epidermidis, S. intermedius, coagulase neg. Staphylococcus or P. multocida; and dental or mouth infections in dogs and oats related to infection by Alcaligenes spp., Bacteroides spp., Clostridium spp., Enterobacter spp., Eubacterium spp., Peptostreptococcus spp., Porphfyromonas spp., Campylobacter spp., Actinomyces spp., Erysipelothrix spp., Rhodococcus spp., Trypanosoma spp., Plasmodium spp., Babesia spp., Toxoplasma spp., Pneumocystis spp., Leishmania spp., and Trichomonas spp. or Prevotella spp. Other bacterial infections and protozoa infections and disorders related to such infections that may be treated or prevented in accord with the method of the present invention are referred to in J. P. Sanford et al., “The Sanford Guide To Antimicrobial Therapy,” 26th Edition, (Antimicrobial Therapy, Inc., 1996).

Antibacterial Activity.

Susceptibility tests can be used to quantitatively measure the in vitro activity of an antimicrobial agent against a given bacterial isolate. Compounds are tested for in vitro antibacterial activity by a micro-dilution method. Minimal Inhibitory Concentration (MIC) is determined in 96 well microtiter plates utilizing the appropriate broth medium for the observed bacterial isolates. Antimicrobial agents are serially diluted (2-fold) in DMSO to produce a concentration range from about 64 μg/ml to about 0.03 μg/ml. The diluted compounds (2 μl/well) are then transferred into sterile, uninoculated medium (0.2 mL) by use of a 96 fixed tip-pipetting station. The inoculum for each bacterial strain is standardized to approximately 5×10⁵ CFU/mL by optical comparison to a 0.5 McFarland turbidity standard. The plates are inoculated with 10 μl/well of adjusted bacterial inoculum. The 96 well plates are covered and incubated at 35+/−2° C. for 24 hours in ambient air environment. Following incubation, plate wells are visually examined by Optical Density measurement for the presence of growth (turbidity). The lowest concentration of an antimicrobial agent at which no visible growth occurs is defined as the MIC. The compounds of the invention generally demonstrated an MIC in the range from about 64 μg/ml to about 0.03 μg/ml.

All in vitro testing follows the guidelines described in the Approved Standards M7-A7 protocol, published by the Clinical Laboratory Standards Institute (CLSI).

The invention further provides compositions and methods of treating patients suffering from an inflammatory condition comprising administering to a patient in need thereof, a therapeutically effective amount of at least one compound of the invention. Specific examples of inflammatory conditions treatable according to the invention include, but are not limited to, scleritis; epi-scleritis; allergic conjunctivitis; pulmonary inflammatory diseases, particularly cystic fibrosis (CF), asthma, chronic obstructive pulmonary disease (COPD), allergic bronchopulmonary aspergillosis (ABPA), and sarcoidosis; procto-sigmoiditis; allergic rhinitis; arthritis; tendonitis; apthous stomatitis; and inflammatory bowel disease.

The invention further provides compositions and methods for i) prophylactic treatment of those patients susceptible to the symptoms CF including pulmonary infection and inflammation associated with CF, ii) treatment at the initial onset of symptoms of pulmonary infection and inflammation associated with CF, and iii) treatment of ongoing or relapsing symptoms of infection and inflammation associated with CF. In accordance with the invention a compound according to any one of compounds of the invention, is administered to a patient in need of treatment for CF, in amount sufficient to prevent, diminish or eradicate symptoms of CF including chronic pulmonary inflammation and infection.

Pharmaceutical Compositions.

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43,650 by Montgomery, all of which are incorporated herein by reference). A discussion of pulmonary delivery of antibiotics is also found in U.S. Pat. No. 6,014,969, incorporated herein by reference.

According to the methods of treatment of the present invention, bacterial infections, cystic fibrosis and inflammatory conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically exipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The pharmaceutical compositions of this invention can be administered orally to fish by blending said pharmaceutical compositions into fish feed or said pharmaceutical compositions may be dissolved in water in which infected fish are placed, a method commonly referred to as a medicated bath. The dosage for the treatment of fish differs depending upon the purpose of administration (prevention or cure of disease) and type of administration, size and extent of infection of the fish to be treated. Generally, a dosage of 5-1000 mg, preferably 20-100 mg, per kg of body weight of fish may be administered per day, either at one time or divided into several times. It will be recognized that the above-specified dosage is only a general range which may be reduced or increased depending upon the age, body weight, condition of disease, etc. of the fish.

Unless otherwise defined, all technical and scientific terms used herein are accorded the meaning commonly known to one of ordinary skill in the art. All publications, patents, published patent applications, and other references mentioned herein are hereby incorporated by reference in their entirety.

Abbreviations.

Abbreviations which may be used in the descriptions of the scheme and the examples that follow are:
Ac for acetyl;
AIBN for azobisisobutyronitrile;
Bu₃SnH for tributyltin hydride;
CDI for carbonyldiimidazole;
dba for dibenzylidene acetone;
dppb for diphenylphosphino butane;
DBU for 1,8-diazabicyclo[5.4.0]undec-7-ene;
DEAD for diethylazodicarboxylate;
DMAP for dimethylaminopyridine;
DMF for dimethyl formamide;
DPPA for diphenylphosphoryl azide;
EtOAc for ethyl acetate;
IPA for isopropyl alcohol;
MeOH for methanol;
Ms for mesylate or O—SO₂—CF₃;
NaN(TMS)₂ or NaHMDS for sodium bis(trimethylsilyl)amide;
NMMO for N-methylmorpholine N-oxide;
TEA for triethylamine;
THF for tetrahydrofuran;
TPP or PPh₃ for triphenylphosphine;
MOM for methoxymethyl;
Boc for t-butoxycarbonyl;
Bz for benzoyl;
Bn for benzyl;
Ph for phenyl;
POPd for dihydrogen dichlorobis(di-tert-butylphosphinito-κP)palladate(II) (PdCl₂[(t-Bu)₂P(OH)]₂);
TBS for tert-butyl dimethylsilyl; or
TMS for trimethylsilyl.
   Synthetic Methods.

The compounds and processes of the present invention will be better understood in connection with the following synthetic schemes that illustrate the methods by which the compounds of the invention may be prepared.

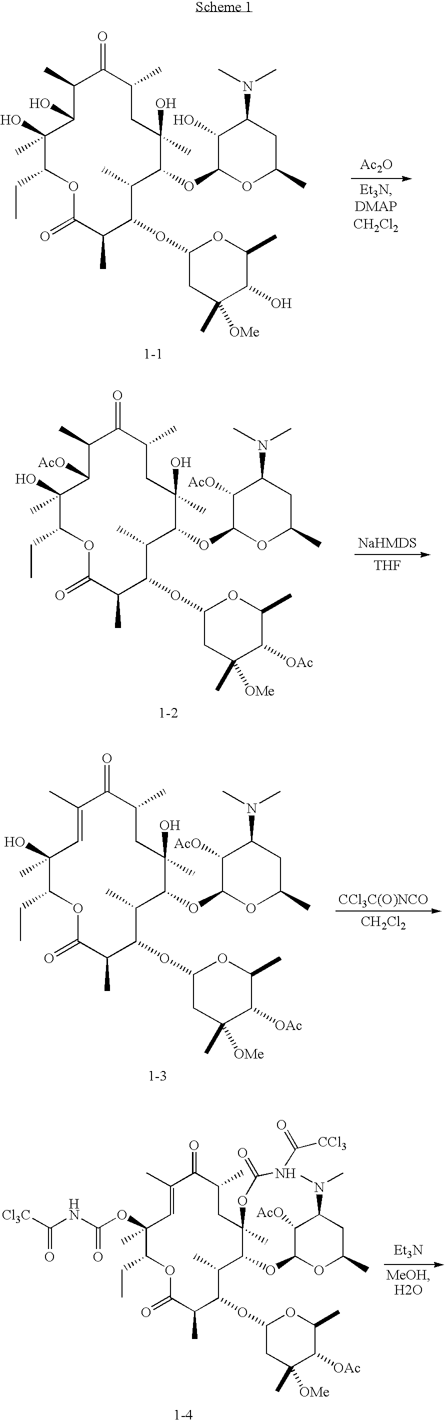

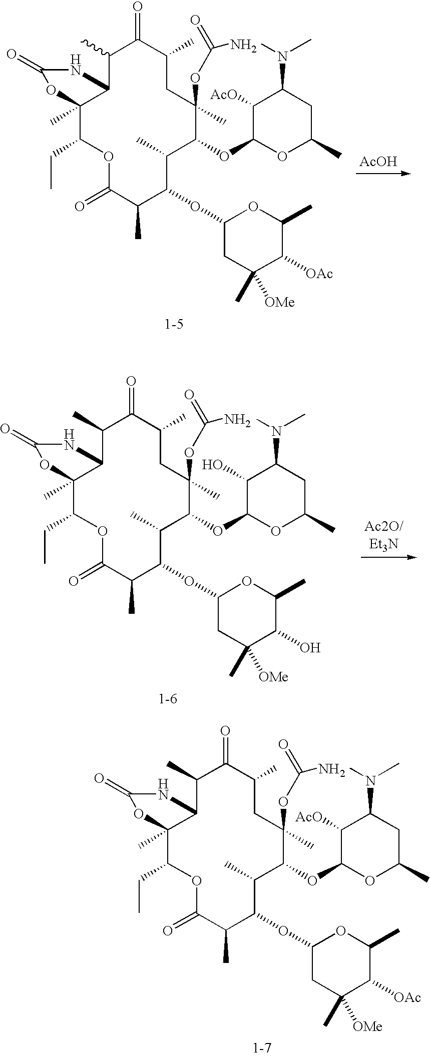

Scheme 1 outlines the synthesis of carbamate intermediate 1-7. Compound 1-1 was acylated with a suitable acylating group such as, but not limited to acetic anhydride to give compound 1-2. Subsequently treatment with base, such as, but not limited to NaHMDS, provided olefin 1-3. Acylation of both the C-6 and C-12 hydroxy groups were achieved with acylating agent such as trichloro acetyl isocyanate to furnish compound 1-4. Removal of the tricloro acetyl group was achieved with basic conditions such as, but not limited to triethylamine in methanol to furnish compound of formula 1-5 as a mixture of isomers with the formation of the cyclic carbamate moiety at C-11 and C-12. Treatment of compound 1-5 with acid such as, but not limited to acetic acid provided compound 1-6 as a single stereoisomer and all the acetyl groups removed. The 2′ hydroxy and the cladinose hydroxy was reprotected with a suitable protecting group such as, but not limited to acetyl to provide compound 1-7.

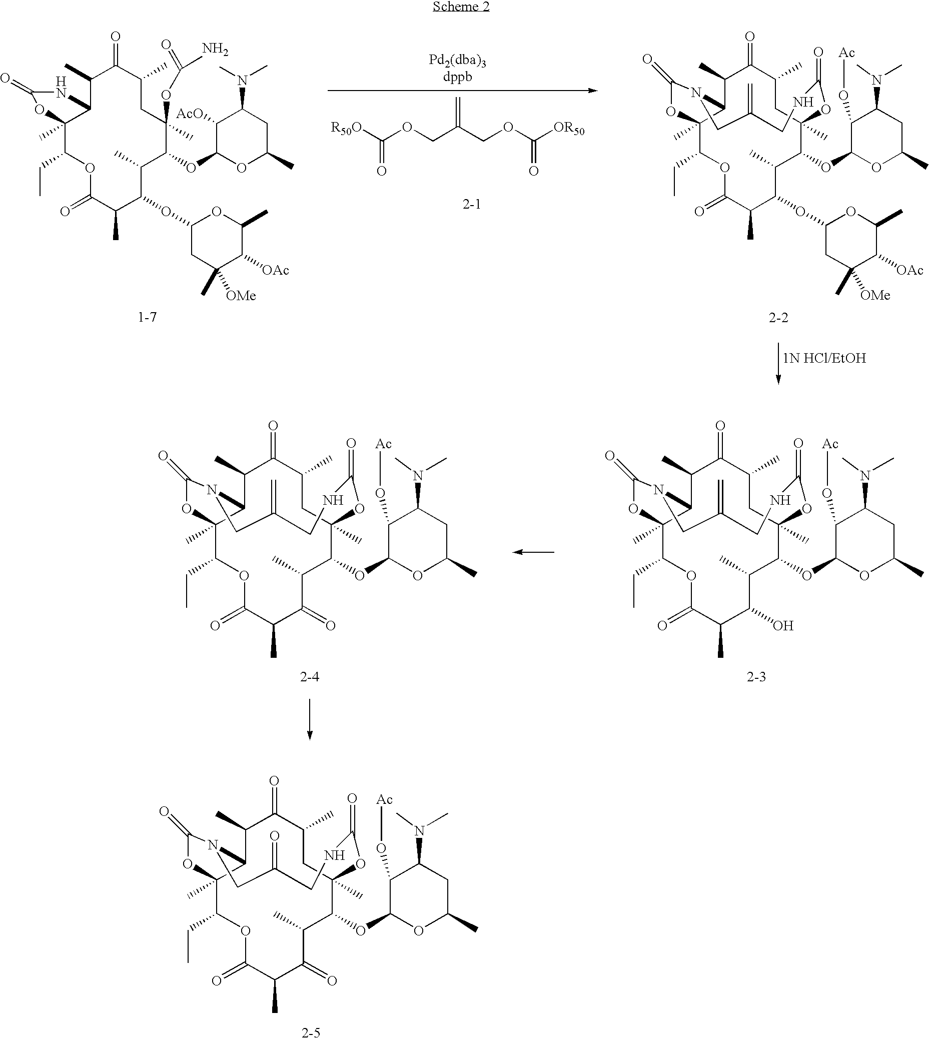

Another process of the invention, as illustrated in Scheme 2, involves preparing a compound of formula (2-5) by reacting a compound of formula (1-7) with a suitable alkylating agent of the formula:

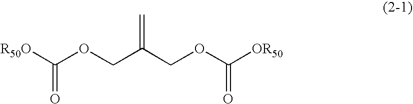

wherein R₅₀ is —C₁-C₁₂ alkyl, —C₁-C₁₂ alkenyl, or —C₁-C₁₂ alkynyl optionally substituted with one or more substitutents selected from halogen, aryl, substituted aryl, heteroaryl, or substituted heteroaryl; —C₃-C₁₂ cycloalkyl. Most palladium (0) catalysts are expected to work in this process. Some palladium (II) catalysts, such as palladium (II) acetate, which is converted into a palladium (0) species in-situ by the actions of a phosphine, will work as well. See, for example, Beller et al. Angew. Chem. Int. Ed. Engl., 1995, 34 (17), 1848. The palladium catalyst can be selected from, but not limited to, palladium (II) acetate, tetrakis(triphenylphospine)palladium (0), tris(dibenzylideneacetone)dipalladium, tetradibenzylideneacetone)dipalladium and the like. Palladium on carbon and palladium (II) halide catalysts are less preferred than other palladium catalysts for this process. Suitable phosphines include, but are not limited to, triphenylphosphine, bis(diphenylphosphino)methane, bis(diphenylphosphino)ethane, bis(diphenylphosphino)propane, 1,4-bis(diphenylphosphino)butane, bis(diphenylphosphino)pentane, and tri-o-tolyl-phosphine, and the like. The reaction is carried out in an aprotic solvent, preferably at elevated temperature, preferably at or above 50° C. Suitable aprotic solvents include, but are not limited to, tetrahydrofuran, N,N-dimethylformamide, dimethyl sulfoxide, N-methyl-2-pyrrolidone, toluene, hexamethylphosphoric triamide, 1,2-dimethoxyethane, methyl-tert-butyl ether, heptane, acetonitrile, isopropyl acetate and ethyl acetate. Generally, the alkylating agents have the formula (2-1) as previously described. The preferred alkylating agents are those wherein R₁₀ is tert-butyl, isopropyl or isobutyl. The alkylating reagents are prepared by reaction of a diol with a wide variety of compounds for incorporating the di-carbonate moiety. The compounds include, but are not limited to, tert-butyl chloroformate, di-tert-butyl dicarbonate, and 1-(tert-butoxycarbonyl)imidazole and the reaction is carried out in the presence of an organic or an inorganic base.

Removal of the cladinose moiety of the macrolide 2-2 either by mild acid hydrolysis or by enzymatic hydrolysis to afford compound 2-3. Representative acids include, but are not limited to, dilute hydrochloric acid, sulfuric acid, perchloric acid, chloroacetic acid, dichloroacetic acid or trifluoroacetic acid. Suitable solvents for the reaction include, but are not limited to, methanol, ethanol, isopropanol, butanol, water and mixtures there of.

Conversion of compounds of formula 2-3 to compounds of formula 2-4 can be accomplished by oxidation of the 3-hydroxy group to a 3-oxo group using Dess-Martin periodinane (for further details concerning the Dess-Martin oxidation see D. B. Dess, J. C. Martin, J. Org. Chem. 48, 4155 (1983)), a Corey-Kim reaction with N-chlorosuccinimide-dimethylsulfide (for further details concerning the Corey-Kim oxidation reaction see E. J. Corey, C. U. Kim, J. Am. Chem. Soc. 94, 7586 (1972)), or a Moffat oxidation with a carbodiimide-DMSO complex in the presence of pyridinium trifluoroacetate, TPAP, PDC, and the like (for further details concerning the Moffat oxidation see J. G. Moffatt, “Sulfoxide-Carbodiimide and Related Oxidations” in Oxidation vol. 2, R. L. Augustine, D. J. Trecker, Eds. (Dekker, New York, 1971) pp 1-64; T. T. Tidwell, Org. React. 39, 297-572 passim (1990); and T. V. Lee, Comp. Org. Syn. 7, 291-303 passim (1991).

Conversion of alkenes (2-4) into ketones (2-5) can be accomplished by ozonolysis followed by decomposition of the ozonide with the appropriate reducing agents. The reaction is typically carried out in an inert solvent such as, but not limited to, methanol, ethanol, ethyl acetate, glacial acetic acid, chloroform, methylene chloride or hexane or mixtures thereof, preferably methanol, preferably at −78° C. to −20° C. Representative reducing agents are, for example, triphenylphosphine, trimethylphosphite, thiourea, and dimethyl sulfide, preferably triphenylphosphine. A more thorough discussion of ozonolysis and conditions therefor may be found in J. March, Advanced Organic Chemistry, 4^{th} ed., Wiley & Son, Inc, 1992. Alternatively, compounds of formula (2-4) can be prepared from compounds of formula (2-5) dihydroxydation with OsO₄ followed by NaIO₄ cleavage.

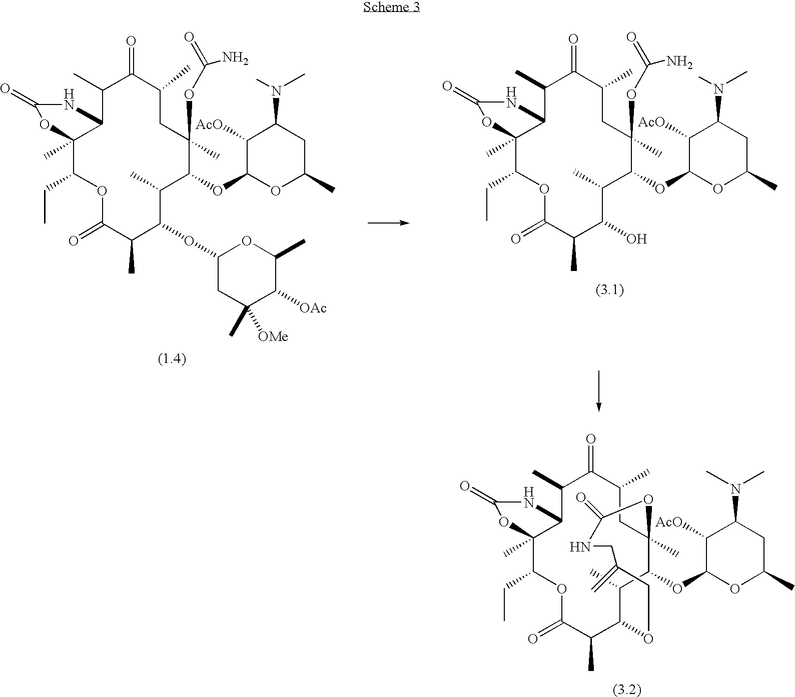

Scheme 3 further illustrates the synthesis of the 3,6 bridged compound 3-2. To obtain the 3,6 carbamate bridge, the cladinose moiety was first removed to give compound 3-1, followed by the alkylation with reagent 2-1 to provide 3-2. The bridge olefin was converted to the corresponding ketone 3-3. Chemistry outlined herewith is similar to the chemistry described in Scheme 2.

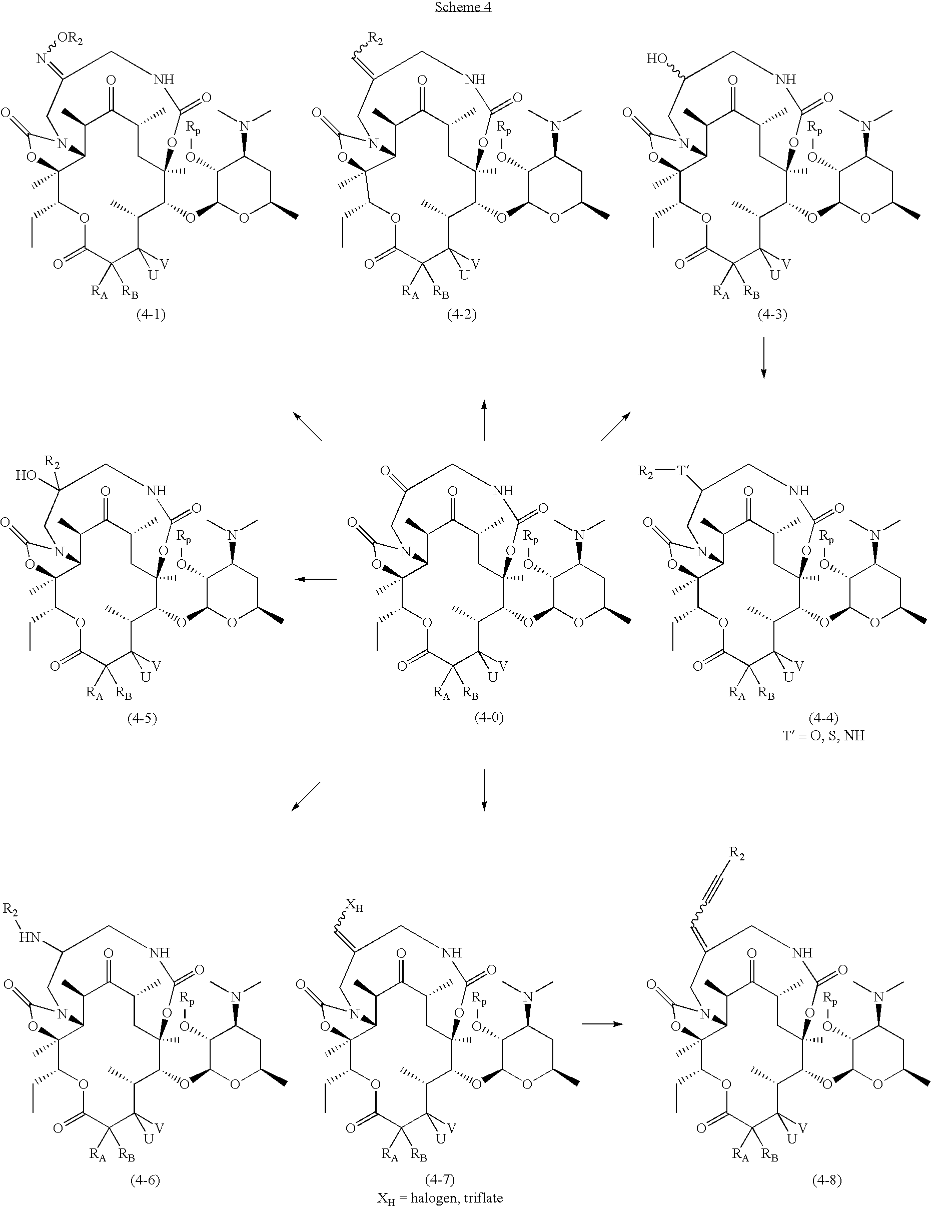

Compounds according to the invention of the formula (4-0) can be further functionalized in a variety of ways. Scheme 4 details a procedure for the conversion of the ketone of formula (4-0) into an oxime of formula (4-1). Oxime formation can be accomplished using the appropriate substituted hydroxylamine under either acidic or basic conditions in a variety of solvents. Representative acids include, but are not limited to, hydrochloric, phosphoric, sulfuric, p-toluenesulfonic, and pyridinium p-toluene sulfonate. Likewise, representative bases include, but are not limited to, triethylamine, pyridine, diisopropylethyl amine, 2,6-lutidine, and the like. Appropriate solvents include, but are not limited to, methanol, ethanol, water, tetrahydrofuran, 1,2-dimethoxyethane, and ethyl acetate. Preferably the reaction is carried out in ethanol using triethylamine as the base.

It will be appreciated by one skilled in the art that ketones of formula (4-0) can be transformed into alkenes of formula (4-2) and (4-7) via Wittig reaction with the appropriate phosphonium salt in the presence of a base, see (a) Burke, Tetrahedron Lett., 1987, 4143-4146, (b) Rathke and Nowak, J. Org. Chem., 1985, 2624-2626, (c) Maryanoff and Reitz, Chem. Rev., 1989, 863-927. Furthermore, vinyl halides of formula (4-7) can be functionalized by Sonogashira coupling with alkynes in the presence of a palladium catalyst, a copper halide and an amine base to give compounds of formula (4-8) (see (a) Sonogashira, Comprehensive Organic Synthesis, Volume 3, Chapters 2,4; (b) Sonogashira, Synthesis 1977, 777.). In a similar manner, alkenes of formula (4-2) can be obtained from vinyl halides (4-7) via Suzuki cross coupling with organoboron reagents in the presence of a palladium catalyst and a base, or via Stille cross coupling with organostananes in the presence of a palladium catalyst (see (a) Suzuki, J. Organomet. Chem. 1999, 576, 147-168, (b) Stille, Angew. Chem. Int. Ed. Engl., 1986, 508-524 (c) Farina, J. Am. Chem. Soc., 1991, 9585-9595).

Furthermore, alcohols of type (4-3) can be prepared by reduction of the corresponding ketone of formula (4-0) under a variety of conditions (see Hudlicky, M. Reductions in Organic Chemistry, Ellis Horwood Limited: Chichester, 1984). The alcohols thus derived can be further modified to give compounds of formula (4-4). A process to generate compounds of formula (4-4) includes, but is not limited to, alkylation of the alcohol with an electrophile or conversion of the alcohol into a leaving group, such as a triflate, tosylate, phosphonate, halide, or the like, followed by displacement with a heteroatom nucleophile (e.g. an amine, alkoxide, sulfide or the like).

Yet another means by which to functionalize ketones of formula (4-0) is via addition of Grignard reagents to form alcohols of formula (4-5). The requisite Grignard reagents are readily available via the reaction of a variety of alkyl or aryl halides with magnesium under standard conditions (see B. S. Furniss, A. J. Hannaford, P. W. G Smith, A. R. Tatchell, Vogel's Textbook of Practical Organic Chemistry, 5^{th} ed., Longman, 1989). The addition is performed in an inert solvent, generally at low temperatures. Suitable solvents include, but are not limited to, tetrahydrofuran, diethylether, 1,4-dioxane, 1,2-dimethoxyethane, and hexanes. Preferably the solvent is tetrahydrofuran or diethylether. Preferably the reaction is run at −78° C. to 0° C.

In a similar way, reaction with other organometallic reagents gives rise to alcohols of formula (4-5). Examples of useful organometallic reagents include, but are not limited to, organo-aluminum, organo-lithium, organo-cerium, organo-zinc, organo-thallium, and organo-boron reagents. A more thorough discussion of organometallic reagents can be found In B. S. Furniss, A. J. Hannaford, P. W. G Smith, A. R. Tatchell, Vogel's Textbook of Practical Organic Chemistry, 5^{th} ed., Longman, 1989.

Ketone of formula (4-0) can be further utilized by conversion into amine of formula (4-6) via a reductive amination. Reductive amination is achieved by treating the ketone with an amine in the presence of a reducing agent to obtain the product amine (4-6). The reaction can be carried out either with or without added acid. Examples of acids that are commonly used include, but are not limited to, hydrochloric, phosphoric, sulfuric, acetic, and the like. Reducing agents that effect reductive amination include, but are not limited to, hydrogen and a catalyst, zinc and hydrochloric acid, sodium cyanoborohydride, sodium borohydride, iron pentacarbonyl, and alcoholic potassium hydroxide. Generally alcoholic solvents are used. The preferred conditions use sodium cyanoborohydride in methanol with added acetic acid.

It will be appreciated by one skilled in the art, that the unsaturated compounds represented by compounds (4-2) and (4-8) can be reduced to form the corresponding saturated compound (see Hudlicky, M., Reductions in Organic Chemistry, Ellis Horwood Limited Chichester, 1984).

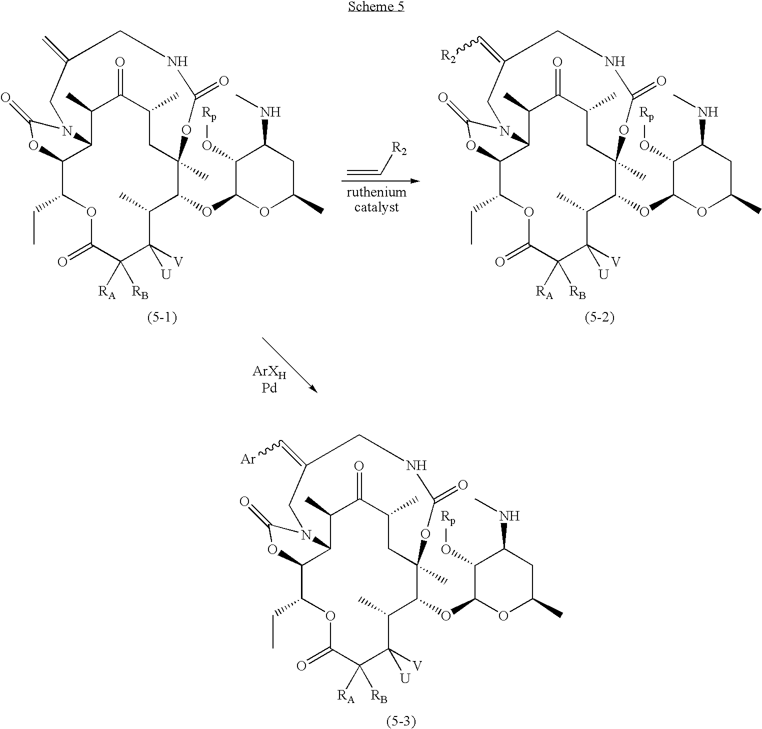

Compounds of the invention according to formula (5-1) are also capable of further functionalization to generate compounds of the present invention. Alkene (5-1) can be treated with an aryl halide or aryl triflate in the presence of a palladium catalyst [Pd(0) or Pd(II)] to provide compound (5-3): (See (a) Heck, Palladium Reagents in Organic Synthesis, Academic Press: New York, 1985, Chapter 1; (b) Sonogashira, Comprehensive Organic Synthesis, Volume 3, Chapters 2,4; (c) Sonogashira, Synthesis 1977, 777). Under the Heck coupling conditions, regioisomers and stereoisomers of the double bond are possible. Alternatively, compound (5-1) can undergo a cross metathesis reaction with vinylaromatic derivatives using ruthenium catalysts to give compounds of formula (5-2) (see (a) J. Org. Chem. 2000, 65, 2204-2207; (b) Reviews: Synlett. 1999, 2, 267; (c) Reviews: Ivin, K. J.; Mol, J. C., Olefin Metathesis and Metathesis Polymerization, 2^{nd} ed., Academic Press: New York, 1997; (d) J. Org. Chem. 1999, 64, 4798-4816; (e) Angew. Chem., Int. Ed. Engl. 1997, 36, 2036-2056; (f) Tetrahedron 1998, 54, 4413-4450).

The chemistry for 6,11-carbamate bridge can also be applied to 3,6-carbamate bridge systems via similar methods illustrated in Schemes 4 and 5.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Example 1

### Compound of Formula III, wherein A and B Taken Together with the Carbon Atom they Attached are C═CH₂, Y═U=hydrogen, V is

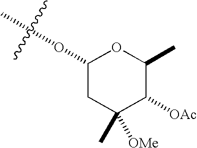

### and Rp=Ac

Step 1a: Compound (1-2)

To a solution of compound (1-1) of Scheme 1 (10 g, 13.64 mmol) in CH₂Cl₂ (27 mL) was added Et₃N (15 mL, 109.12 mmol), acetic anhydride (7.74 mL, 81.86 mmol) and DMAP (167 mg, 1.37 mmol) at room temperature. The reaction mixture was stirred for 3 hours at room temperature. The reaction mixture was diluted with CH₂Cl₂ and quenched with saturated sodium bicarbonate. The resulting mixture was extracted with CH₂Cl₂ (50 mL×3). The combine organic layers were dried (Na₂SO₄) and filtered. The filtrate was concentrated to give compound (1-2) (12.6 g) and was carried into the next step without further purification. MS (ESI) m/z=860.6 (M+H)⁺.

Step 1b: Compound (1-3)

To a solution of compound from step 1a (12.6 g, 13.64 mmol) in anhydrous THF (136 mL), which was cooled to 0° C., added NaHMDS (1.0 M solution in THF, 16.4 mL, 16.4 mmol). The reaction mixture was kept at 0° C. for 3 hours. The resulting reaction mixture was diluted with ethyl acetate (200 mL), washed with brine (150 mL×2), dried (Na₂SO₄), filtered and concentrated to give compound (1-3) (11.4 g). MS (ESI) m/z=800.6 (M+H)⁺.

Step 1c: Compound (1-4)

To a solution of compound from step 1b (8.0 g, 10.01 mmol) in CH₂Cl₂ (100 mL) at 0° C. was added trichloroacetyl isocyanate (3.6 mL, 30.03 mmol) dropwised over 20 min. The resulting mixture was further stirred at 0° C. for 1 hour. To the reaction mixture was added 5 mL of IPA, stirred for 5 min and concentrated under reduced pressure to give compound (1-4) (14 g). MS (ESI) m/z=1174.1, 1176.2, 1178.2. (M+H)⁺.

Step 1d: Compound (1-5)

To a solution of compound from step 1c (14 g, 10.01 mmol) in IPA (80 mL) was added Et₃N (2 mL) and H₂O (7.2 mL) at room temperature. The reaction mixture was refluxed for 4 hours then evaporated to dryness. The resulting mixture was dissolved in Ethyl acetate (200 mL), washed with saturated NaHCO₃ (100 mL×2) and brine solution (100 mL). The organic layers were dried (Na₂SO₄), and concentrated under reduced pressure to afford 8.8 g of Compound (1-5). MS (ESI) m/z=886.5 (M+H)⁺.

Step 1e: Compound (1-6)

A solution of compound from step 1d (2.0 g, 2.73 mmol) in 5% of AcOH-EtOH (22 mL) was heated to reflux for 17 hours. The resulting mixture was concentrated under reduced pressure to give 1.66 g of Compound (1-6). MS (ESI) m/z=844.53 (M+H)⁺.

Step 1f: Compound (1-7)

To a solution of compound from Step 1e in CH₂Cl₂ (20 mL) was added triethyl amine (0.55 mL, 3.94 mmol) and acetic anhydride (0.37 mL, 3.94 mmol). The reaction mixture was stirred at room temperature for 12 hours. The resulting reaction mixture was diluted with CH₂Cl₂ (100 mL) and quenched with saturated NaHCO₃, the organic layers were dried (Na₂SO₄), filtered and filtrate was concentrated and purified by flash chromatography (silica gel, Hexanes/Acetone=5/1 to 1/1) to give 900 mg of Compound (1-7). MS (ESI) m/z=886.5 (M+H)⁺.

Step 1g: Compound (2-2)

A solution of compound from Step 1f (500 mg, 0.565 mmol) in THF (20 mL) was added Pd₂(dba)₃ (259 mg, 0.282 mmol), dppb (239 mg, 0.565 mmol) and allyl-bis Boc (prepared as 0.5M solution in THF, 2.24 mL, 1.12 mmol). The reaction mixture was de-gassed in high vacuum (×2) then refluxed for 1.5 hours. The resulting mixture was filtered, concentrated in vacuo and the crude mixture was purified by flash chromatography (silica gel, acetone/hexane=1/2) to give 200 mg of the title compound (not pure), which was re purified on HPLC (reverse phase, 60% CH₃CN-40% NH₄HCO₃) to give 80 mg of the titled compound. MS (ESI) m/z=938.8 (M+H)⁺.

### Example 2

### Compound of Formula III, wherein A and B Taken Together with the Carbon Atom they Attached are C═CH₂, Y═U=hydrogen, V═OH, and Rp=Ac

To a solution of compound from step 1g of Example 1 (270 mg, 0.29 mmol) in Ethanol (8 mL) was added 2N HCl (4 mL) and the resulting mixture was stirred at the room temperature for 3 hours. The resulting mixture was cooled to 0° C. and was added H₂O (8 mL) and extracted with diethyl ether (20 mL×2). The aqueous layer was diluted with CH₂Cl₂ (20 mL), neutralized with saturated NaHCO₃, separated organic layers. The aqueous layer was further extracted with the CH₂Cl₂ (20 mL×2). The combined organic layers were concentrated and purified by flash chromatography (silica gel, Hexanes/Acetone=5/1 to 1/1) giving 150 mg of the titled compound. MS (ESI) m/z=738.7 (M+H)⁺.

### Example 3

### Compound of Formula III wherein A and B Taken Together with the Carbon Atom they Attached are C═CH₂, U and V Taken Together with the Carbon Atom they Attached are C═O, and Rp=Ac

A mixture of compound from Example 2 (150 mg, 0.19 mmol), HOAc (0.018 mL) and Dess-Martin periodinane (173 mg, 0.407 mmol) in CH₂Cl₂ (10 mL) was stirred at room temperature for 2 hours. The reaction mixture was quenched with saturated NaHCO₃ (5 mL) and resulting mixture was washed with 0.5 N NaOH (10 mL). The organic phase was dried (Na₂SO₄), filtered and filtrate was concentrated to give 150 mg of the titled compound. MS (ESI) m/z=736.7 (M+H)⁺.

### Example 4

### Compound of Formula III wherein A and B Taken Together with the Carbon Atom they Attached are C═O, U and V Taken Together with the Carbon Atom they Attached are C═O, and Rp=Ac

To a solution of compound from Example 3 (30 mg, 0.041 mmol) in methanol (10 mL), which was cooled to −78° C., was added CSA (19 mg, 0.082 mmol). The reaction mixture was passed through O₂ for 5 min then generated O₃ until the colorless solution turned to blue (about 10 min). The resulting mixture was added Me₂S (1 mL) at −78° C. then warmed to room temperature for 1 hr. The reaction mixture was quenched with saturated NaHCO₃ and extracted with CH₂Cl₂ (5 mL×3). The combined organic layers were dried (Na₂SO₄) and filtered. The organic solvent was removed under reduced pressure to give 40 mg of the titled compound. MS (ESI) m/z=738.4(M+H)⁺.

### Example 5

### Compound of Formula IV, wherein A and B Taken Together with the Carbon Atom they Attached are C═O, U and V Taken Together with the Carbon Atom they Attached are C═O, and Rp=Ac

Step 5a: Compound (3-1)

A mixture of compound from step 1f of Example 1 (2.4 g, 2.73 mmol) in ethanol (16 mL) and 2N HCl (11 mL) was stirred at the room temperature for 48 hours. The resulting mixture was diluted with CH₂Cl₂ (120 mL) then neutralized with saturated 3N NaOH, organic layers were separated and the aqueous layer was further extracted with CH₂Cl₂ (100 mL×2). The combined organic layers were dried (Na₂SO₄), filtered and filtrate was concentrated and purified by flash chromatography (silica gel, Hexanes/Acetone=5/1 to 1/1) giving 1.87 g (100%) of Compound (3-1). MS (ESI) m/z=686.8 (M+H)⁺.

Step 5b: Compound (3-2)

A solution of compound from step 5a of Example 5 (200 mg, 0.292 mmol) in THF (10 mL) was added Pd₂(dba)₃ (107 mg, 0.117 mmol), P(OiPr)₃ (Triisopropyl phosphite) (0.99 mL, 4.0 mmol) and ally-bis Boc (prepared as 0.5M solution in THF, 1.2 mL, 0.584 mmol). The reaction mixture was de-gassed then heated to reflux for 1.5 hours. The resulting mixture was cooled to room temperature, filtered and concentrated in vacuo. The crude mixture was purified by flash chromatography (silica gel, acetone/hexane=1/10 to 1/1) to give 80 mg of the crude product, which was purified on HPLC (reverse phase, 45% CH₃CN-55% NH₄HCO₃) to give 50 mg of the titled compound. MS (ESI) m/z=738.5 (M+H)⁺.

1. Compounds represented by formulae (1) and (II): 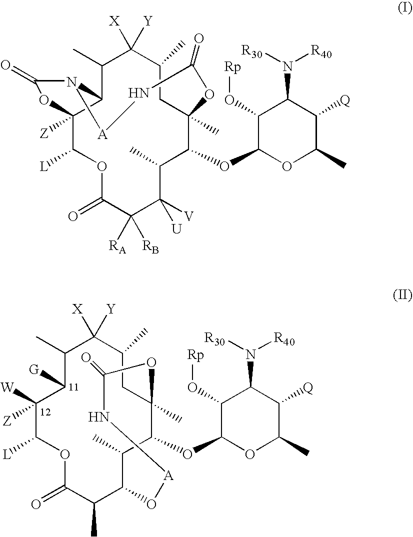
or the pharmaceutically acceptable salts, esters and prodrugs thereof,
wherein A is:
(a) —R₁—, where R₁ is substituted or unsubstituted —C₁-C₈ alkylene-, —C₂-C₈ alkenylene- or —C₂-C₈ alkynylene-, containing 0, 1, 2, or 3 heteroatoms selected from O, S or N;
(b) —R₁—(C═O)—R₂—, where R₂ is independently selected from R₁;
(c) —R₁—(C═N-M-R₃)—R₂—, where M is absent, O, NH, NH(CO), NH(CO)NH or NHSO₂; and R₃ is independently selected from the group consisting of:
(1) hydrogen;
(2) aryl; substituted aryl; heteroaryl; substituted heteroaryl; and
(3) R₄, where R₄ is substituted or unsubstituted —C₁-C₆ alkyl, —C₂-C₆ alkenyl, or —C₂-C₆ alkynyl containing 0, 1, 2, or 3 heteroatoms selected from O, S or N; and
(4) substituted or unsubstituted, saturated or unsaturated C₃-C₁₂ cycloalkyl containing 0, 1, 2, or 3 heteroatoms selected from O, S or N;
(d) —R₁—[C(OR₅)(OR₆)]—R₂—, where R₅ and R₆ are selected from the group consisting of C₁-C₁₂ alkyl, aryl or substituted aryl; or taken together —(CRxRy)ₘ-, where m is 2 or 3, Rx and Ry are independently R₃, alternatively, Rx and Ry can be taken together to form a fused heterocyclic or heteroaromatic ring;
(e) —R₁—[C(SR₅)(SR₆)]—R₂—; or
(f) —R₁—(C═CH—R₃)—R₂—;
X and Y are each independently selected from the group consisting of:
(a) hydrogen;
(b) halogen;
(c) protected hydroxyl;
(d) —OC(O)R₃;
(e) —OR₃; and
(f) —NR₇R₈; wherein R₇ and R₈ are each independently selected from R₃; or R₇R₈ taken with the nitrogen atom to which they are connected form a 3 to 7-membered ring which may optionally contain a hetero function selected from the group consisting of —O—, —NR₃—, —S—, —S(O)—, and —S(O)₂—;
alternatively, X and Y taken together with the carbon atom to which they are attached is:
(a) C═O;
(b) C═N—OR₉, wherein R₉ is selected from the group consisting of:
(1) hydrogen;
(2) —CH₂—O—(CH₂)₂OCH₃;
(3) —CH₂—O—(CH₂O)ₙCH₃, wherein n is as previously defined;
(4) —R₄;
(5) substituted and unsubstituted, saturated or unsaturated C₃-C₁₂ cycloalkyl;
(6) substituted and unsubstituted heterocyclic;
(7) C(O)—(C₃-C₁₂ cycloalkyl);
(8) C(O)—R₃, wherein R₃ is as previously defined;
(9) —Si(Rₐ)(R_{b})(R_{c}), wherein Rₐ, R_{b} and R_{c} are each independently selected from the group consisting of C₁-C₁₂ alkyl, aryl and substituted aryl; or
(10) C═N—O—C(R₉)(R₁₀)—O—R₁₁ wherein R₉ and R₁₀ taken together with the carbon atom to which they are attached form a C₃ to C₁₂ cycloalkyl group or each independently is selected from the group consisting of: hydrogen and C₁-C₁₂ alkyl; and R₁₁ is selected from the group consisting of:
(i) —R₄;
(ii) substituted and unsubstituted, saturated or unsaturated —C₃-C₁₂ cycloalkyl; and
(iii) —Si(Rₐ)(R_{b})(R_{c}), wherein Rₐ, R_{b} and R_{c} are as previously defined;
one U or V is hydrogen and the other is independently:
a) hydrogen;
b) hydroxyl;
c) protected hydroxyl;
d) —R₄; wherein R₄ is as previously defined
e) —OR₃;
f) —C(O)R₃;
g) —OC(O)R₃;
h) —S(O)ₙR₃; or
i) 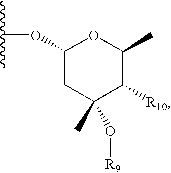
where R₉ is selected from the group consisting of hydrogen and methyl; and R₁₀ is:
i. hydrogen;
ii. hydroxyl or hydroxyl protecting group;
iii. —R₄; or
iv. —OR₄;
alternatively, U and V taken together with the carbon atom to which they are attached to form a carbonyl group;
each of R_{A} and R_{B} is independently:
a) hydrogen;
b) halogen; or
c) —R₄;
W is selected from the group consisting of:
a) hydrogen;
b) —R₄; and
c) —OR₄;
G is —OR₃; where R₃ is as previously defined; alternatively, 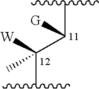
moiety of formula (II) taken together is:
a)
b) 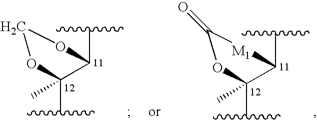
where M₁ is O or N-J-R₂₀, and where J is absent, O, NH, NH(CO), or N═CH; and R₂₀ is independent selected from R₃;
R₃₀ and R₄₀ is independently selected from the group consisting of hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, saturated or unsaturated heterocyclic group; or can be taken together with the nitrogen atom to which they are attached to form a substituted or unsubstituted heterocyclic or heteroaromatic ring;
Z is selected from the group consisting of hydrogen, azido, cyano, nitro, aldehyde, carboxylic acid, amide, a substituted or unsubstituted, saturated or unsaturated aliphatic group;
Q is selected from the group consisting of:
(a) hydrogen;
(b) protected hydroxy; and
(c) OR₂₁, where R₂₁ is selected from the group consisting of:
(i) —R₄; and
(ii) substituted or unsubstituted, saturated or unsaturated —C₃-C₁₂ cycloalkyl containing 0, 1, 2, or 3 heteroatoms selected from O, S or N;
L is —R₄;
Rp is hydrogen, hydroxy protecting group or hydroxy prodrug group.

2. A compound according to claim 1 represented by formula (III): 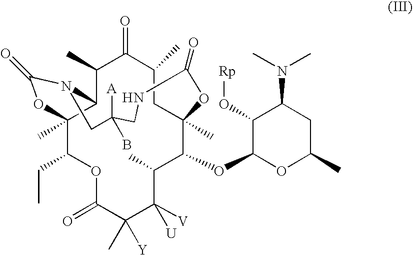
wherein A and B are independently selected from the group consisting of:
a) hydrogen;
b) hydroxyl;
c) activated hydroxyl;
d) N₃;
e) NH₂;
f) CN;
g) protected hydroxyl;
h) protected amino;
i) -T-R₃, where T is absent, O, S, S(O), SO₂, NH, NCH₃, NH(CO), NH(CO)NH or NHSO₂; and R₃ is as previously defined; and
j) 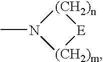
wherein E is absent, O, S, S(O), S(O)₂, NR₃, N(CO)R₃, NSO₂R₃, or CHR₃; n=1, 2, or 3; and m=2 or 3;
alternatively, A and B taken together with the carbon atom to which they are attached is:
a) C═O;
b) C(OR₅)(OR₆)], where R₅ and R₆ are selected from the group consisting of C₁-C₁₂ alkyl, aryl or substituted aryl; or taken together —(CRxRy)ₘ-, where m is 2 or 3, Rx and Ry are independently R₃, alternatively, Rx and Ry can be taken together to form a fused heterocyclic or heteroaromatic ring;
c) C(SR₅)(SR₆);
d) C═CHR₃;
e) C═NRₐₚ; where Rₐₚ is amino protecting group; or
f) C═N-M-R₃, where M is absent, O, NH, NH(CO), NH(CO)NH or NHSO₂;
and U, V, Y, and Rₚ are as previously defined in claim 1.

3. A compound according to claim 1 represented by formula (IV): 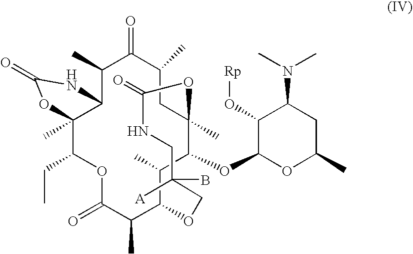
where A, B, and Rₚ are as previously defined in claim 1.

4. A compound according to claim 1 represented by formula (V): 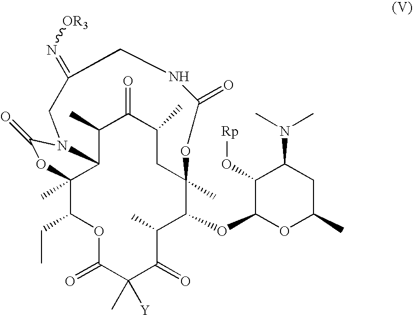
where Y, R₃ and Rₚ are as previously defined in claim 1.

5. A compound according to claim 1 represented by formula (VI): 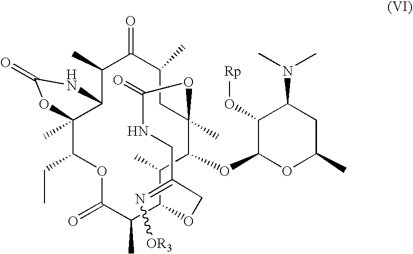
where R₃ and Rₚ are as previously defined in claim 1.

6. A compound of claim 2 selected from:
(a) Compound of Formula III, wherein A and B taken together with the carbon atom they attached are C═CH₂, Y═U=hydrogen, V is 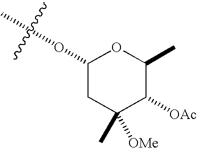
and Rp=Ac;
(b) Compound of Formula III, wherein A and B taken together with the carbon atom they attached are C═CH₂, Y═U=hydrogen, V═OH, and Rp=Ac;
(c) Compound of Formula III, wherein A and B taken together with the carbon atom they attached are C═CH₂, U and V taken together with the carbon atom they attached are C═O, and Rp=Ac;
(d) Compound of Formula III, wherein A and B taken together with the carbon atom they attached are C═O, U and V taken together with the carbon atom they attached are C═O, and Rp=Ac.

7. A compound of claim 3, wherein A and B taken together with the carbon atom they attached are C═O, U and V taken together with the carbon atom they attached are C═O, and Rp=Ac.

8. A compound of claim 1 having the Formula A, wherein X and Y are taken together with the carbon to which they are attached (CXY), selected from compounds 6-17 of Table 1:
TABLE 1

| | |
|---|---|
| | (A) |
| 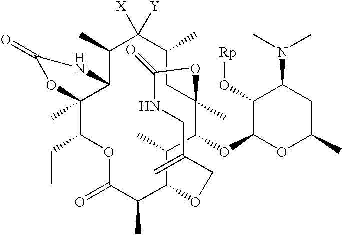 | |
| Compound | CXY | Rp |
|---|---|---|
| (6) | C═NOAc | Ac |
| (7) | C═NOBz | Bz |
| (8) | C═NOSiEt₃ | SiEt₃ |
| (9) | 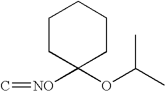 | Ac |
| (10) | 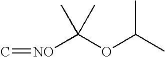 | Ac |
| (11) | C═NOH | H |
| (12) | C═NH | H |
| (13) | CH(NH₂) | H |
| (14) | C═O | H |
| (15) | C═O | Ac |
| (16) | C═NAc | Ac |
| (17) | C═NAc | H |

9. A compound of claim 1 having the Formula B, wherein A and B are taken together with the carbon to which they are attached (CAB), selected from compounds 18-165 of Table 2:
TABLE 2

| | |
|---|---|
| | (B) |
| 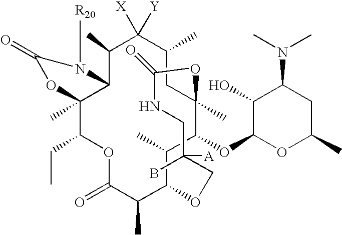 | |
| Compound | R₂₀ | CAB |
|---|---|---|
| (18) | H | C═O |
| (19) | H | C═O |
| (20) | 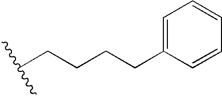 | C═CH₂ |
| (21) | 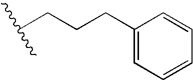 | C═CH₂ |
| (22) | 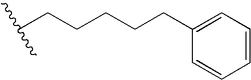 | C═CH₂ |
| (23) | 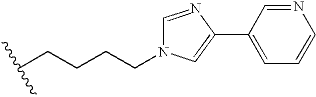 | C═CH₂ |
| (24) | 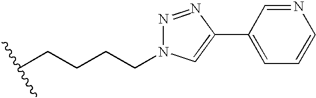 | C═CH₂ |
| (25) | 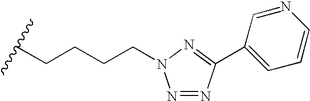 | C═CH₂ |
| (26) | 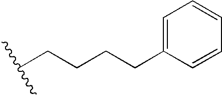 | C═O |
| (27) | 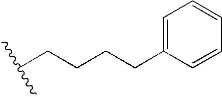 | CHCH₃ |
| (28) | 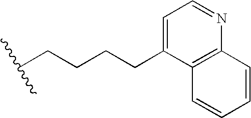 | C═CH₂ |
| (29) | H | C═NO[CH₂Ph] |
| (30) | H | C═NO[(CH₂)₂Ph] |
| (31) | H | C═NO[(CH₂)₃Ph] |
| (32) | H | C═NO[(CH₂)₄Ph] |
| (33) | H | C═NO[Ph] |
| (34) | H | C═NO[(CH₂)₅Ph] |
| (35) | H | 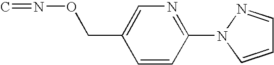 |
| (36) | H | 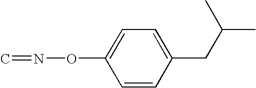 |
| (37) | H | 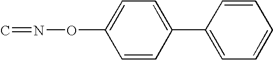 |
| (38) | H | 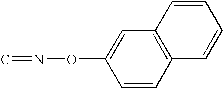 |
| (39) | H | 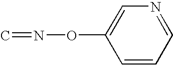 |
| (40) | H | C═NNH[Ph] |
| (41) | H | CH(CH₂)₃Ph |
| (42) | H | CHCH₃ |
| (43) | H | CHOH |
| (44) | H | 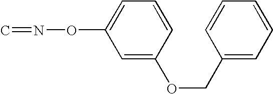 |
| (45) | H | 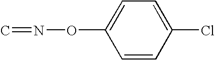 |
| (46) | H | 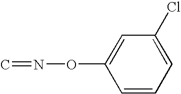 |
| (47) | H | 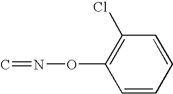 |
| (48) | H | 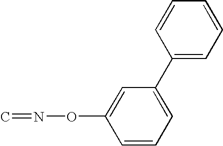 |
| (49) | H | 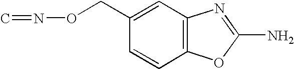 |
| (50) | H | 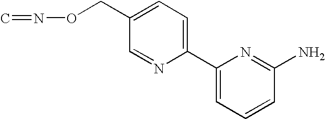 |
| (51) | 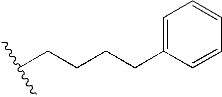 | C═NO [Ph] |
| (52) | 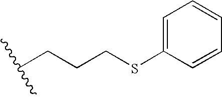 | C═CH₂ |
| (53) | 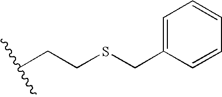 | C═CH₂ |
| (54) | H | 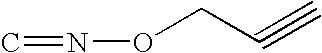 |
| (55) | H | 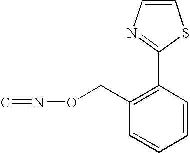 |
| (56) | H | 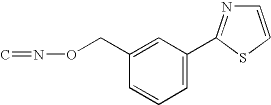 |
| (57) | H | 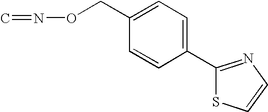 |
| (58) | H | 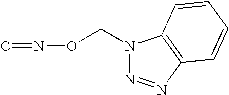 |
| (59) | H | 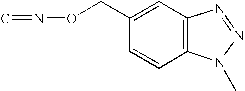 |
| (60) | H | 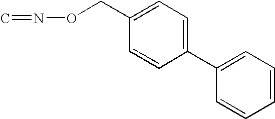 |
| (61) | H | 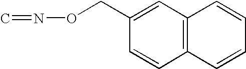 |
| (62) | H | 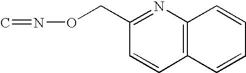 |
| (63) | H | 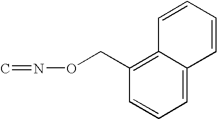 |
| (64) | H | 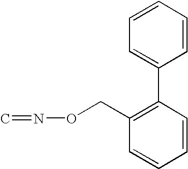 |
| (65) | H | 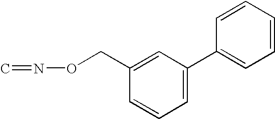 |
| (66) | H | 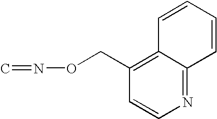 |
| (67) | H | 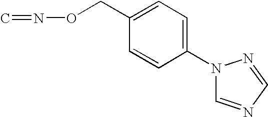 |
| (68) | H | 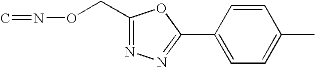 |
| (69) | H | 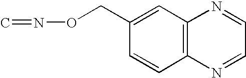 |
| (70) | H | 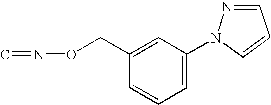 |
| (71) | H | 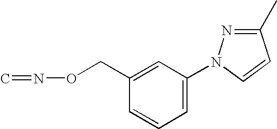 |
| (72) | H | 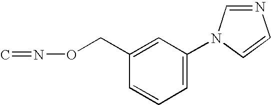 |
| (73) | H | 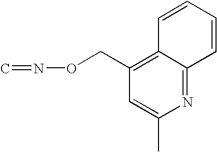 |
| (74) | H | 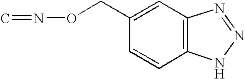 |
| (75) | H | 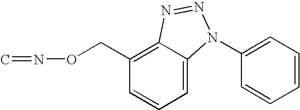 |
| (76) | H | 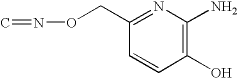 |
| (77) | H | 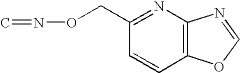 |
| (78) | H | 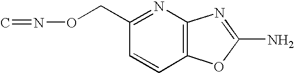 |
| (79) | H | 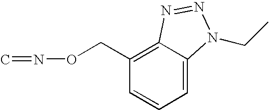 |
| (80) | H | C═N—OH |
| (81) | H | 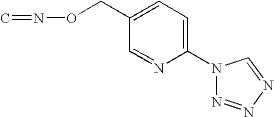 |
| (82) | H | 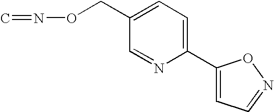 |
| (83) | H | 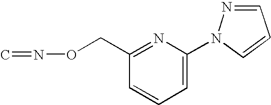 |
| (84) | H | 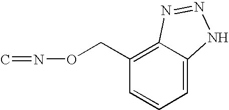 |
| (85) | H | 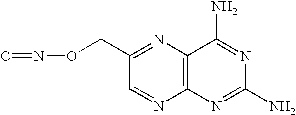 |
| (86) | H | 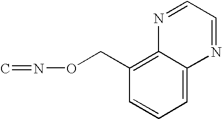 |
| (87) | H | 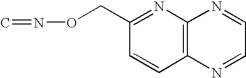 |
| (88) | H | 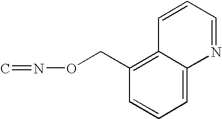 |
| (89) | H | 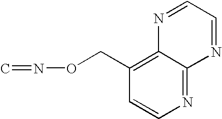 |
| (90) | H | 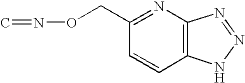 |
| (91) | H | 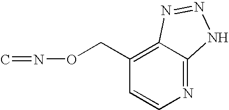 |
| (92) | H | 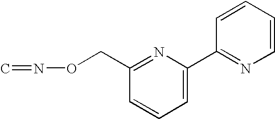 |
| (93) | H | 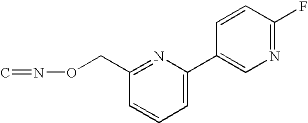 |
| (94) | H | 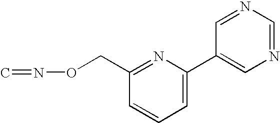 |
| (95) | H | 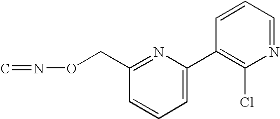 |
| (96) | H | 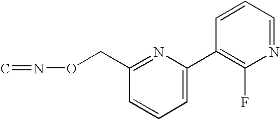 |
| (97) | H | 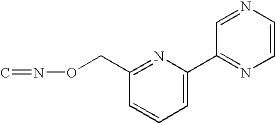 |
| (98) | H | 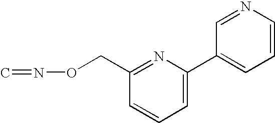 |
| (99) | H | 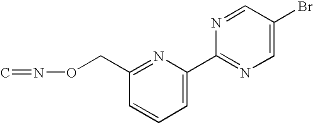 |
| (100) | H | 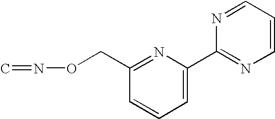 |
| (101) | H | 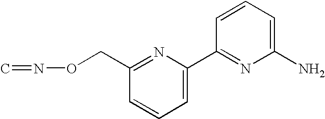 |
| (102) | H | 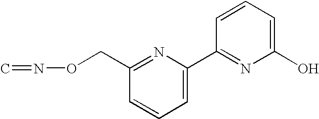 |
| (103) | H | 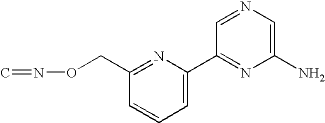 |
| (104) | H | 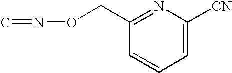 |
| (105) | H | 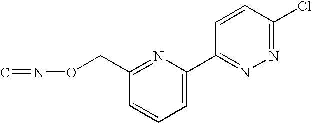 |
| (106) | H | 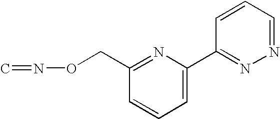 |
| (107) | H | 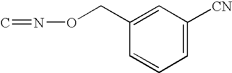 |
| (108) | H | 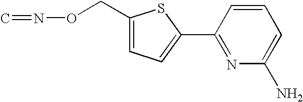 |
| (109) | H | 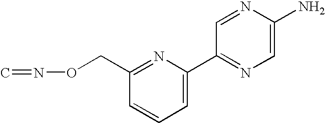 |
| (110) | H | 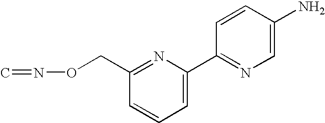 |
| (111) | H | 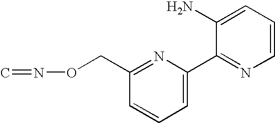 |
| (112) | H | 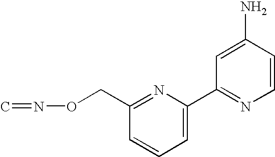 |
| (113) | H | 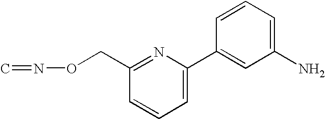 |
| (114) | H | 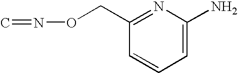 |
| (115) | H | 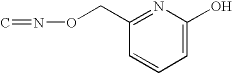 |
| (116) | H | 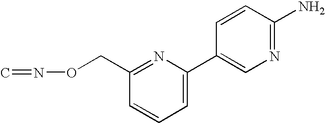 |
| (117) | H | 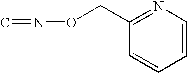 |
| (118) | H | 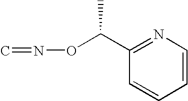 |
| (119) | H | 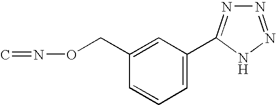 |
| (120) | CH₃ | C═CH₂ |
| (121) | CH₃ | C═O |
| (122) | CH₃ | 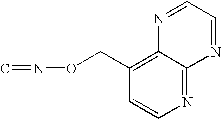 |
| (123) | CH₃ | 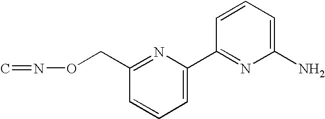 |
| (124) | H | 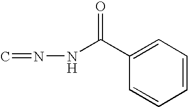 |
| (125) | H | 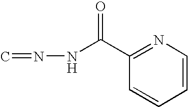 |
| (126) | H | 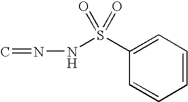 |
| (127) | H | 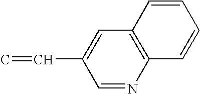 |
| (128) | H | 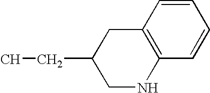 |
| (129) | H | 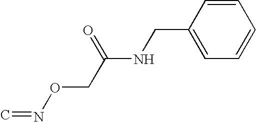 |
| (130) | H | 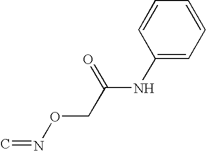 |
| (131) | H | 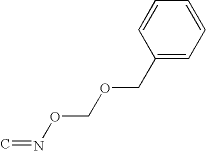 |
| (132) | H | 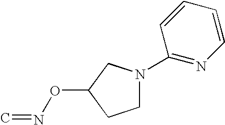 |
| (133) | H | 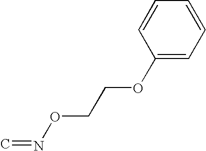 |
| (134) | H | 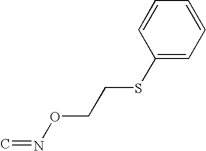 |
| (135) | H | 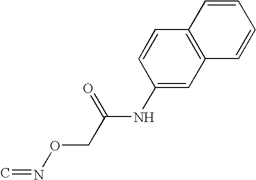 |
| (136) | H | 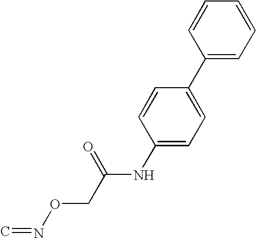 |
| (137) | H | 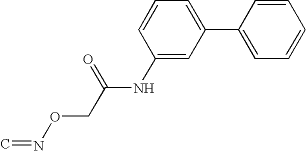 |
| (138) | H | 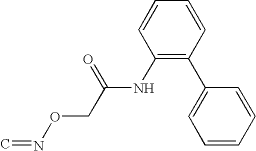 |
| (139) | H | 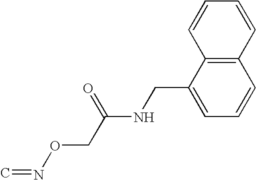 |
| (140) | H | 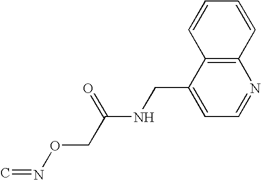 |
| (141) | H | 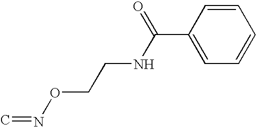 |
| (142) | H | 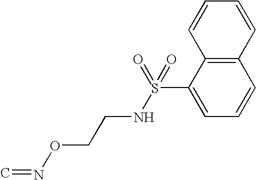 |
| (143) | H | 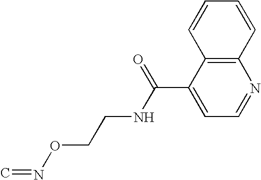 |
| (144) | H | 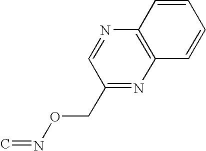 |
| (145) | H | 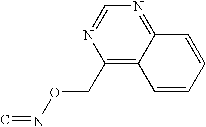 |
| (146) | H | 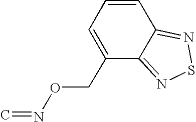 |
| (147) | H | 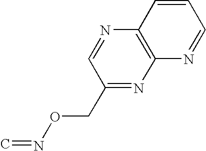 |
| (148) | H | 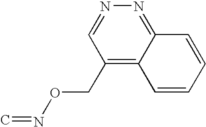 |
| (149) | H | 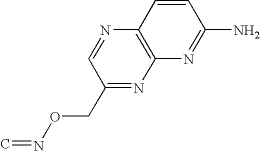 |
| (150) | H | 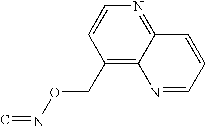 |
| (151) | H | 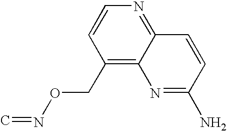 |
| (152) | H | 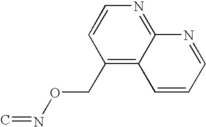 |
| (153) | H | 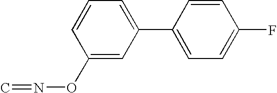 |
| (154) | H | 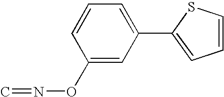 |
| (155) | H | 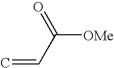 |
| (156) | H | 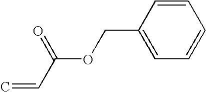 |
| (157) | H | 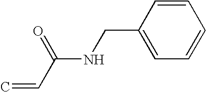 |
| (158) | H | 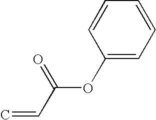 |
| (159) | H | 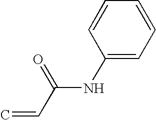 |
| (160) | H | 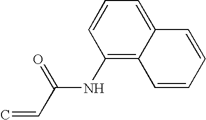 |
| (161) | H | 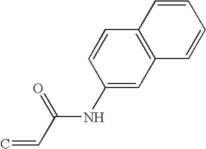 |
| (162) | H | 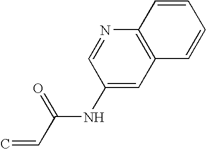 |
| (163) | H | 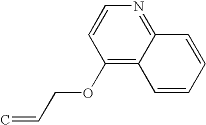 |
| (164) | H | 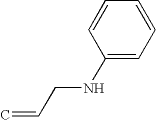 |
| (165) | H | 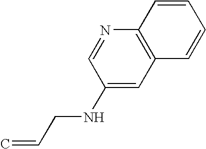 |

10. A compound of claim 1 having the Formula B, selected from compounds 166-232 of Table 3:
TABLE 3

| | |
|---|---|
| | (B) |
| 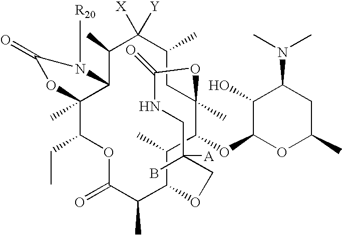 | |
| Compound | R₂₀ | A | B |
|---|---|---|---|
| (166) | H | 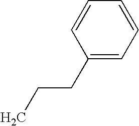 | OH |
| (167) | H | 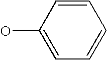 | H |
| (168) | H | 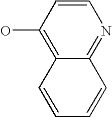 | H |
| (169) | H | 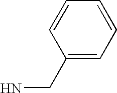 | H |
| (170) | H | 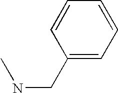 | H |
| (171) | H | 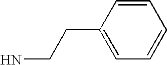 | H |
| (172) | H | 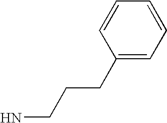 | H |
| (173) | H | 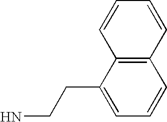 | H |
| (174) | H | 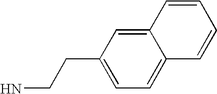 | H |
| (175) | H | 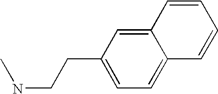 | H |
| (176) | H | 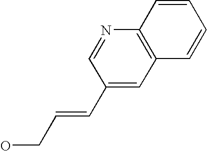 | H |
| (177) | H | 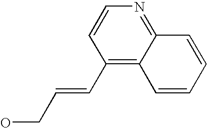 | H |
| (178) | H | OH (R-) | H |
| (179) | H | 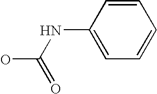 | H |
| (180) | H | 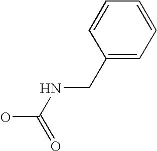 | H |
| (181) | H | OSO₂CH₃ (S-) | H |
| (182) | H | N₃ | H |
| (183) | H | NH₂ | H |
| (184) | H | 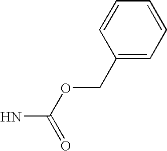 | H |
| (185) | H | 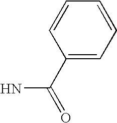 | H |
| (186) | H | 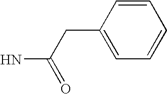 | H |
| (187) | H | 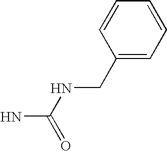 | H |
| (188) | H | 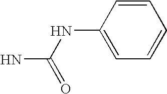 | H |
| (189) | H | 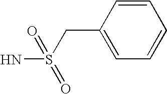 | H |
| (190) | H | 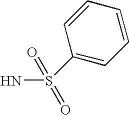 | H |
| (191) | H | 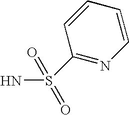 | H |
| (192) | H | 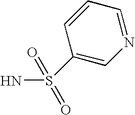 | H |
| (193) | H | 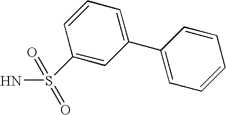 | H |
| (194) | H | 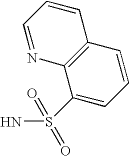 | H |
| (195) | H | 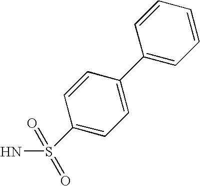 | H |
| (196) | H | 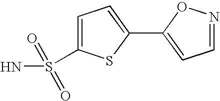 | H |
| (197) | H | 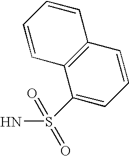 | H |
| (198) | H | 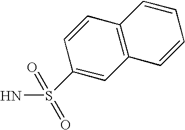 | H |
| (199) | H | 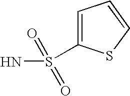 | H |
| (200) | H | 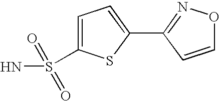 | H |
| (201) | H | 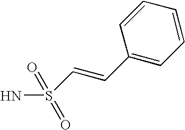 | H |
| (202) | H | 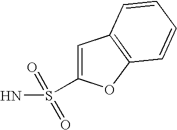 | H |
| (203) | H | 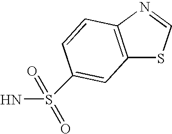 | H |
| (204) | H | 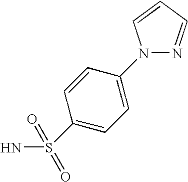 | H |
| (205) | H | 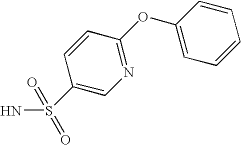 | H |
| (206) | H | 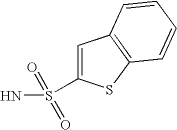 | H |
| (207) | H | 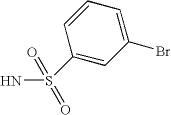 | H |
| (208) | H | 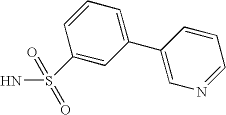 | H |
| (209) | H | 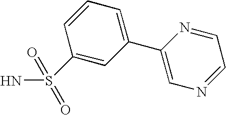 | H |
| (210) | H | 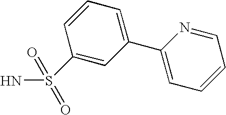 | H |
| (211) | H | 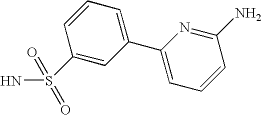 | H |
| (212) | H | 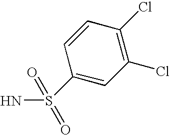 | H |
| (213) | H | 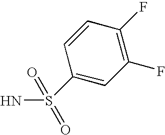 | H |
| (214) | H | 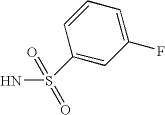 | H |
| (215) | H | 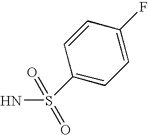 | H |
| (216) | H | 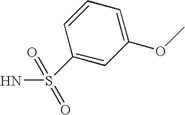 | H |
| (217) | H | 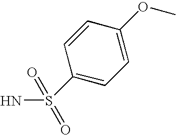 | H |
| (218) | H | 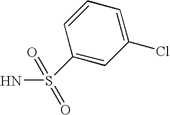 | H |
| (219) | H | 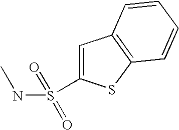 | H |
| (220) | H | 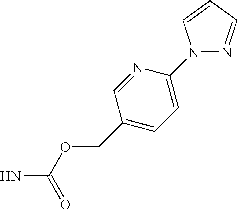 | H |
| (221) | H | 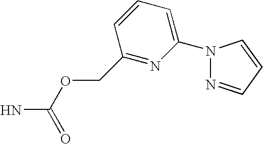 | H |
| (222) | H | 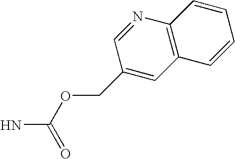 | H |
| (223) | H | 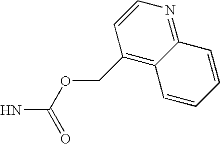 | H |
| (224) | H | 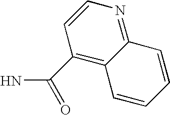 | H |
| (225) | H | 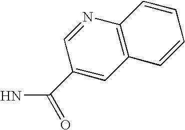 | H |
| (226) | H | 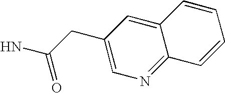 | H |
| (227) | H | 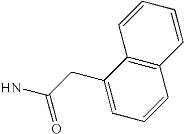 | H |
| (228) | H | 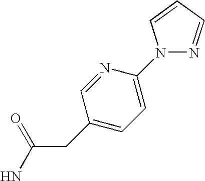 | H |
| (229) | H | 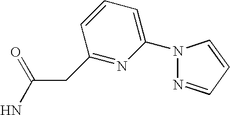 | H |
| (230) | H | 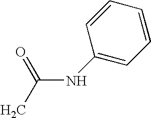 | H |
| (231) | H | 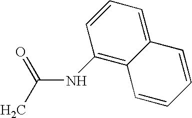 | H |
| (232) | H | 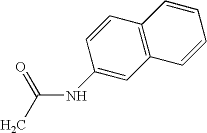 | H |

11. A compound of claim 1 having the Formula C, selected from compounds 233-394 of Table 4:
TABLE 4

| | |
|---|---|
| | (C) |
| 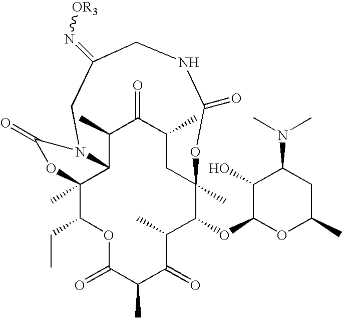 | |
| Compound | R₃ |
|---|---|
| (233) | 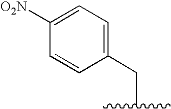 |
| (234) | 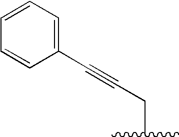 |
| (235) | 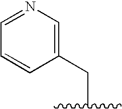 |
| (236) | 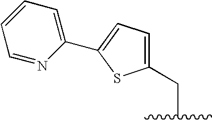 |
| (237) | 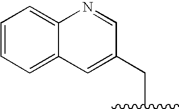 |
| (238) | 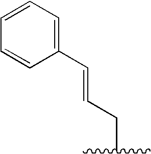 |
| (239) | 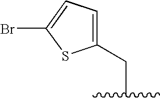 |
| (240) | 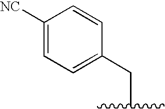 |
| (241) | 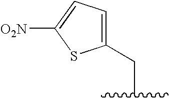 |
| (242) | 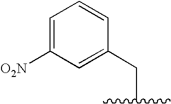 |
| (243) | 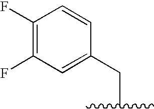 |
| (244) | 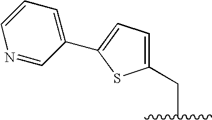 |
| (245) | 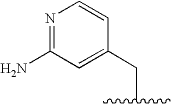 |
| (246) | 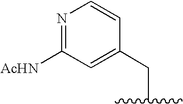 |
| (247) | 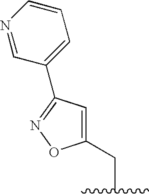 |
| (248) | 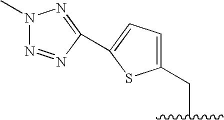 |
| (249) | 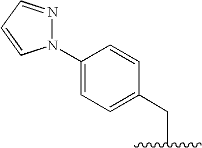 |
| (250) | 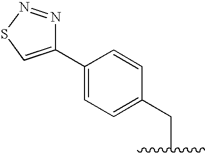 |
| (251) | 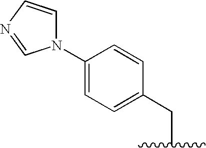 |
| (252) | 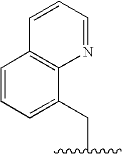 |
| (253) | 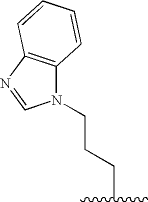 |
| (254) | 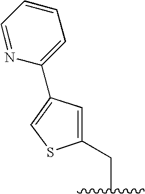 |
| (255) | 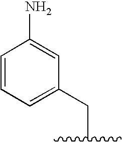 |
| (256) | 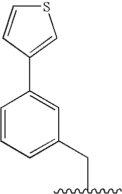 |
| (257) | 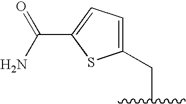 |
| (258) | 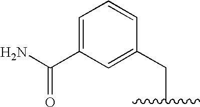 |
| (259) | 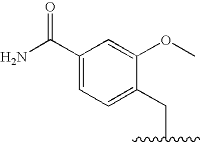 |
| (260) | 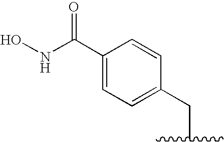 |
| (261) | 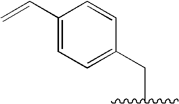 |
| (262) | 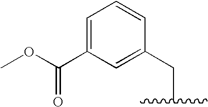 |
| (263) | 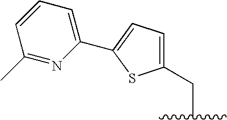 |
| (264) | 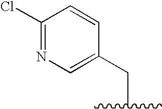 |
| (265) | 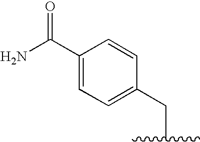 |
| (266) | 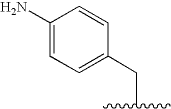 |
| (267) | 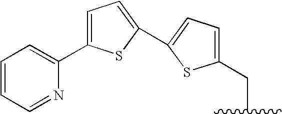 |
| (268) | 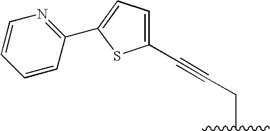 |
| (269) | 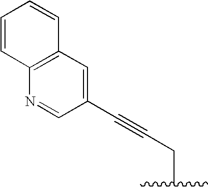 |
| (270) | 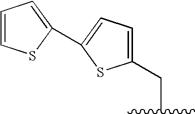 |
| (271) | 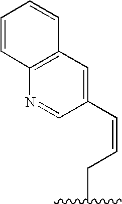 |
| (272) | 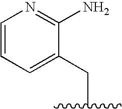 |
| (273) | 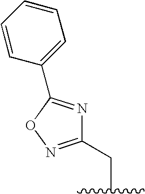 |
| (274) | 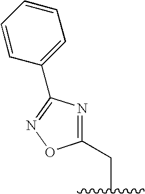 |
| (275) | 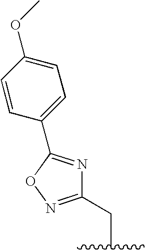 |
| (276) | 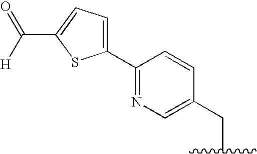 |
| (277) | 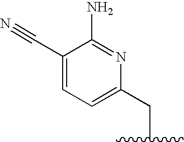 |
| (278) | 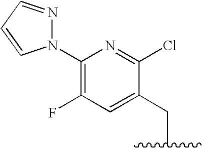 |
| (279) | 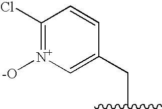 |
| (280) | 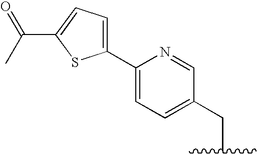 |
| (281) | 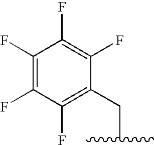 |
| (282) | 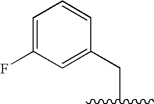 |
| (283) | 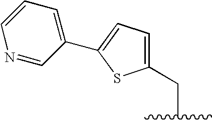 |
| (284) | 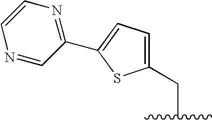 |
| (285) | 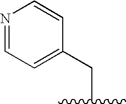 |
| (286) | 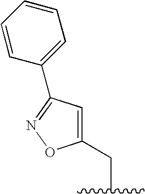 |
| (287) | 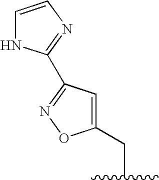 |
| (288) | 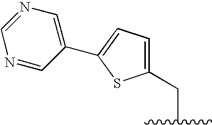 |
| (289) | 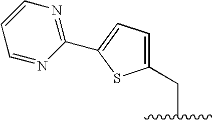 |
| (290) | 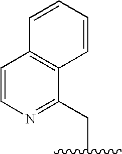 |
| (291) | 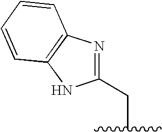 |
| (292) | 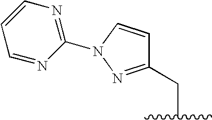 |
| (293) | 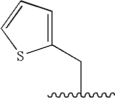 |
| (294) | 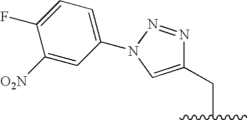 |
| (295) | 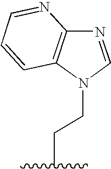 |
| (296) | 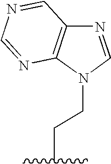 |
| (297) | 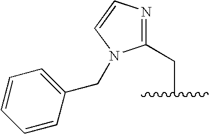 |
| (298) | 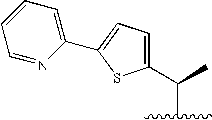 |
| (299) | 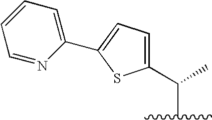 |
| (300) | 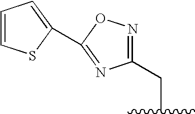 |
| (301) | 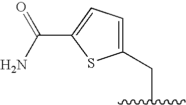 |
| (302) | 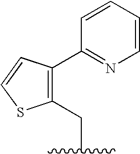 |
| (303) | 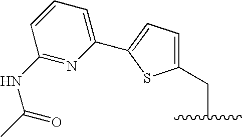 |
| (304) | 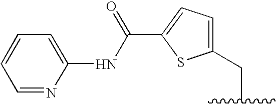 |
| (305) | 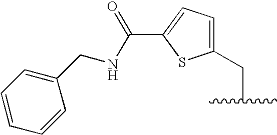 |
| (306) | 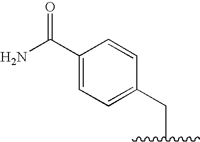 |
| (307) | 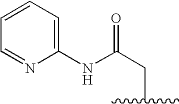 |
| (308) | 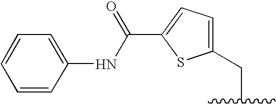 |
| (309) | 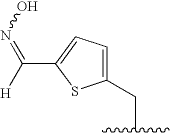 |
| (310) | 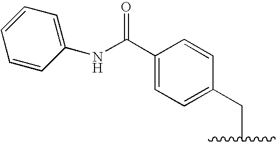 |
| (311) | 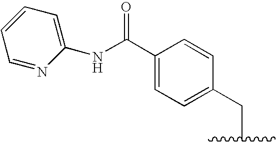 |
| (312) | 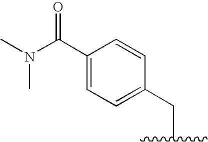 |
| (313) | 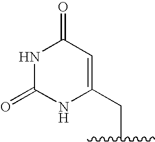 |
| (314) | 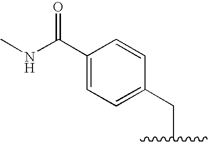 |
| (315) | 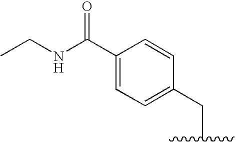 |
| (316) | 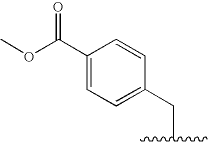 |
| (317) | 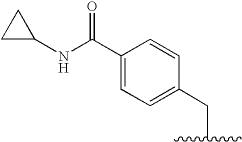 |
| (318) | 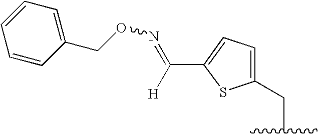 |
| (319) | 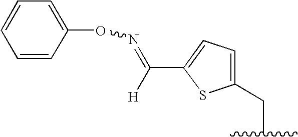 |
| (320) | 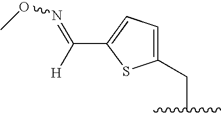 |
| (321) | 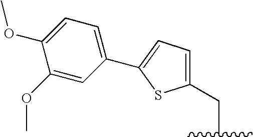 |
| (322) | 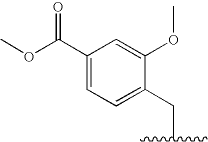 |
| (323) | 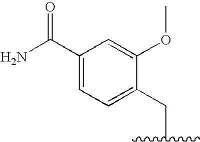 |
| (324) | 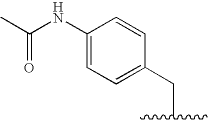 |
| (325) | 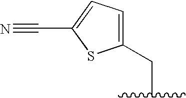 |
| (326) | 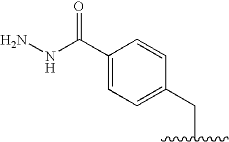 |
| (327) | 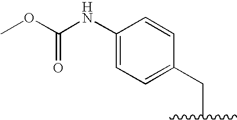 |
| (328) | 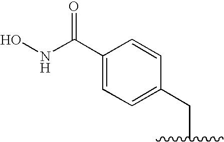 |
| (329) | 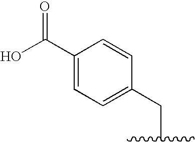 |
| (330) | 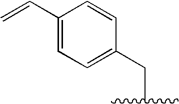 |
| (331) | 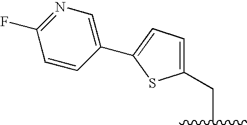 |
| (332) | 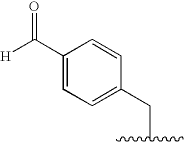 |
| (333) | 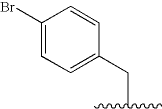 |
| (334) | 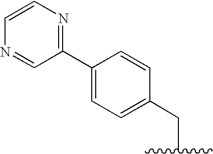 |
| (335) | 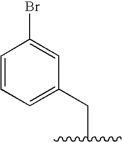 |
| (336) | 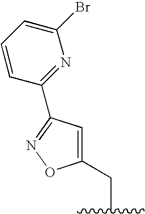 |
| (337) | 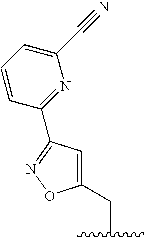 |
| (338) | 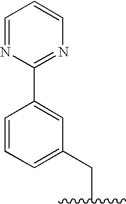 |
| (339) | 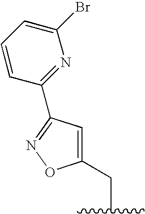 |
| (340) | 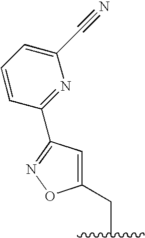 |
| (341) | 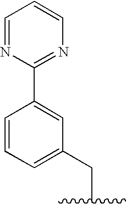 |
| (342) | 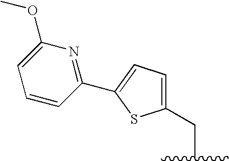 |
| (343) | 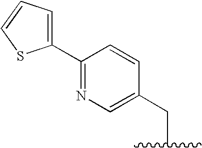 |
| (344) | 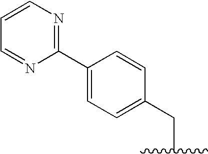 |
| (345) | 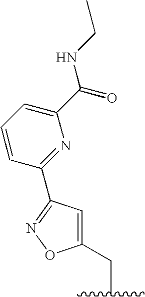 |
| (346) | 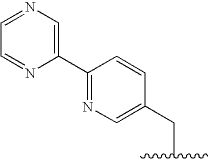 |
| (347) | 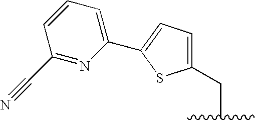 |
| (348) | 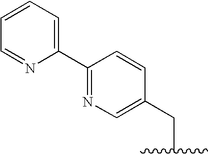 |
| (349) | 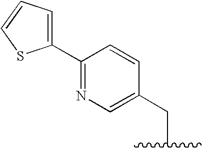 |
| (350) | 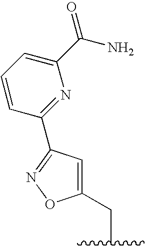 |
| (351) | 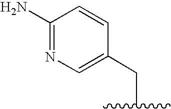 |
| (352) | 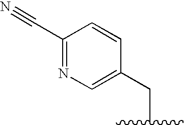 |
| (353) | 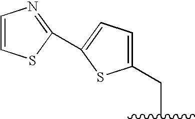 |
| (354) | 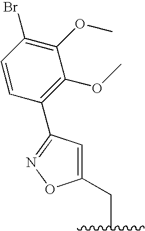 |
| (355) | 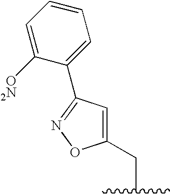 |
| (356) | 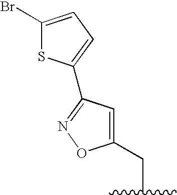 |
| (357) | 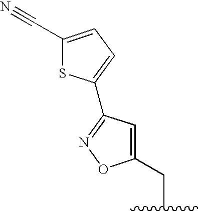 |
| (358) | 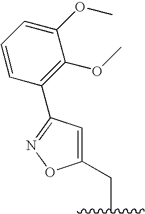 |
| (359) | 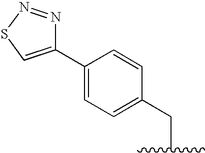 |
| (360) | 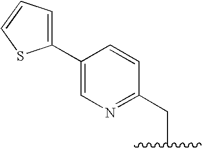 |
| (361) | 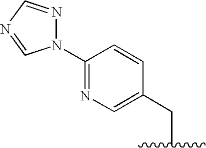 |
| (362) | 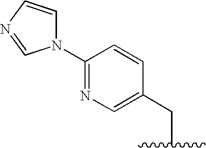 |
| (363) | 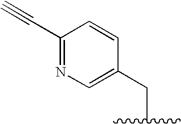 |
| (364) | 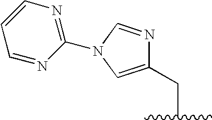 |
| (365) | 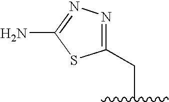 |
| (366) | 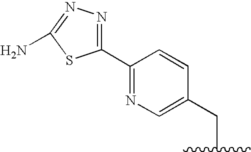 |
| (367) | 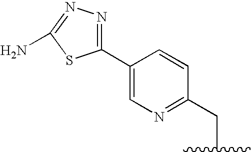 |
| (368) | 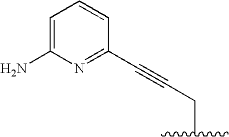 |
| (369) | 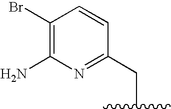 |
| (370) | 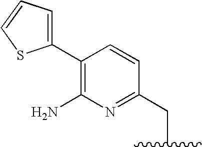 |
| (371) | 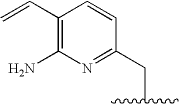 |
| (372) | 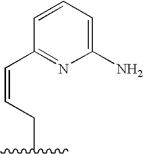 |
| (373) | 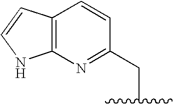 |
| (374) | 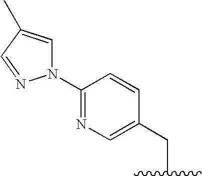 |
| (375) | 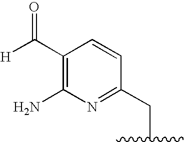 |
| (376) | 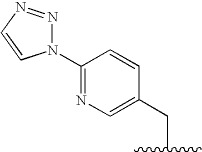 |
| (377) | 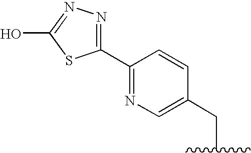 |
| (378) | 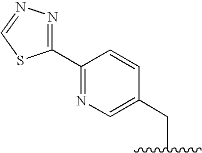 |
| (379) | 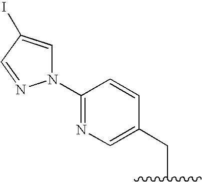 |
| (380) | 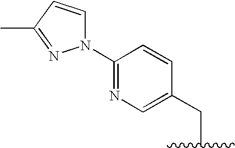 |
| (381) | 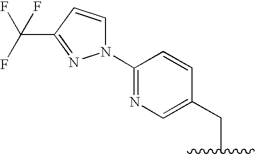 |
| (382) | 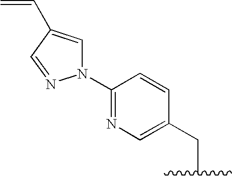 |
| (383) | 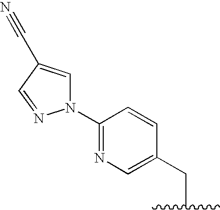 |
| (384) | 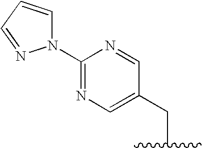 |
| (385) | 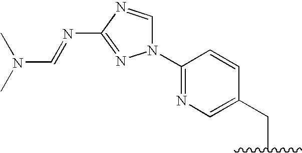 |
| (386) | 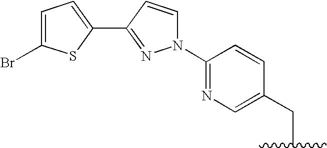 |
| (387) | 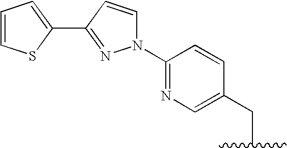 |
| (388) | 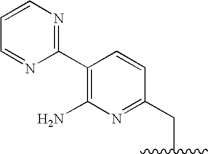 |
| (389) | 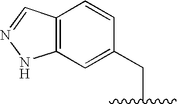 |
| (390) | 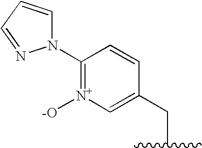 |
| (391) | 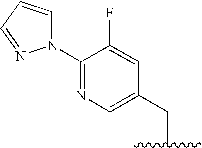 |
| (392) | 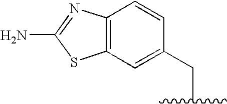 |
| (393) | 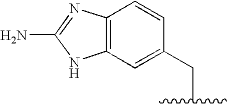 |
| (394) | 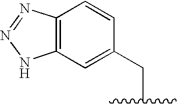 |

12. (canceled)

13. A pharmaceutical composition comprising a therapeutically effective amount of a compound of claim 1 or a pharmaceutically acceptable salt, ester or prodrug thereof, in combination with a pharmaceutically acceptable carrier.

14. A method for treating a bacterial infection in a subject, comprising administering to said subject a therapeutically effective amount of a pharmaceutical composition according to claim 13.

15. A method of treating cystic fibrosis in subject, comprising administering to said subject, a therapeutically effective amount of a pharmaceutical composition of claim 13.

16. A method of treating inflammation in a subject comprising administering to said subject, therapeutically effective amount of a pharmaceutical composition of claim 13.

17. A process for producing a compound of claim 1 having the formula: 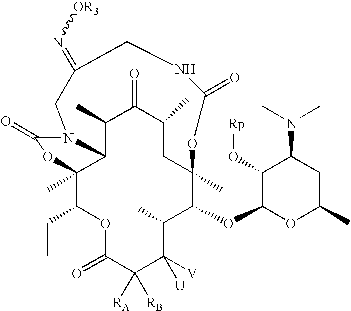
comprising the steps of:
(a) reacting a compound having a formula: 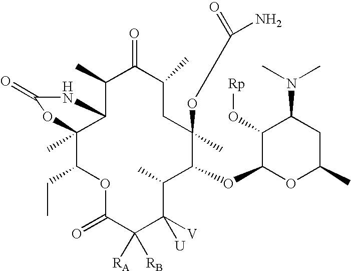
with 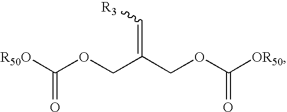
in the presence of a phosphine ligand and Pd(0) catalyst under room temperature to reflux conditions;
(b) oxidative cleavage of the compounds with the following formula: 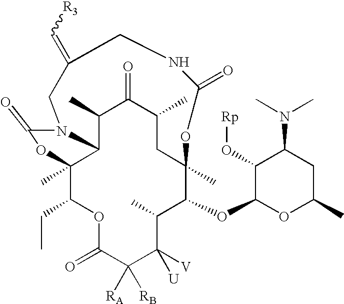
with an oxidizing reagent to give compounds of the following formula: 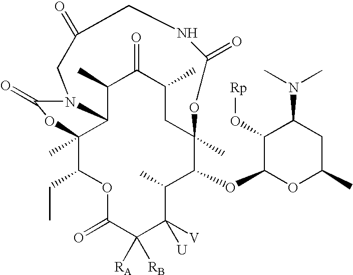
and
(c) reacting the compounds prepared in step (b) with R₃ONH₂, in a presence of a mild acid;
(d) deprotecting the 2′ protecting group with a mild acid.

18. A process for producing a compound of claim 5 having the formula: 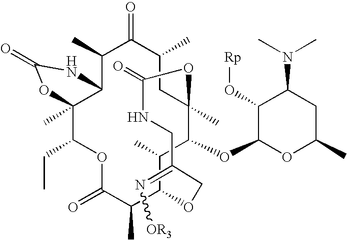
comprising the steps of:
(a) reacting a compound having a formula: 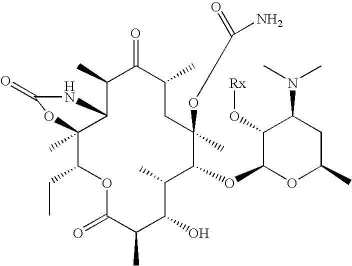
with 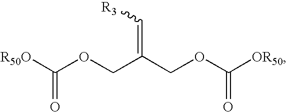
in the presence of a phosphine ligand and Pd(0) catalyst under room temperature to reflux conditions;
(b) oxidative cleavage of the compounds with the following formula: 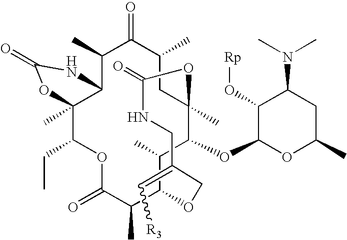
with an oxidizing reagent to give compounds of the following formula: 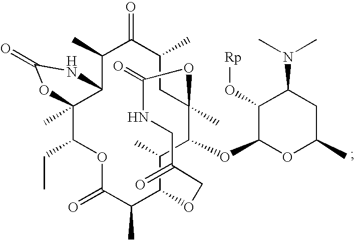
and
(c) reacting the compounds prepared in step (b) with R₃ONH₂, in a presence of a mild acid;
(d) deprotecting the 2′ protecting group with a mild acid.
